(19) United States
(12) Patent Application Publication
(10) Pub. No.: US 2021/0358577 A1
(43) Pub. Date: Nov. 18, 2021

(54) VARIATIONAL AUTO ENCODER FOR MIXED DATA TYPES

(71) Applicant: Microsoft Technology Licensing, LLC, Redmond, WA (US)

(72) Inventors: Cheng ZHANG, Cambridge (GB); Chao MA, Cambridge (GB); Richard Eric TURNER, Cambridge (GB); José Miguel HERNÁNDEZ LOBATO, Cambridge (GB); Sebastian TSCHIATSCHEK, Vienna (AT)

(21) Appl. No.: 16/996,348

(22) Filed: Aug. 18, 2020

(30) Foreign Application Priority Data
May 7, 2020 (GB) 2006809.4

(51) Int. Cl. G16H 10/20; G06N 20/00; G06N 5/02

(52) U.S. Cl. CPC G16H 10/20; G06N 5/02; G06N 20/00

(57) ABSTRACT

In a first stage, training each of a plurality of first variational auto encoders, VAEs, each comprising: a respective first encoder arranged to encode a respective subset of one or more features of a feature space into a respective first latent representation, and a respective first decoder arranged to decode from the respective latent representation back to a decoded version of the respective subset of the feature space, wherein different subsets comprise features of different types of data. In a second stage following the first stage, training a second VAE comprising: a second encoder arranged to encode a plurality of inputs into a second latent representation, and a second decoder arranged to decode the second latent representation into decoded versions of the first latent representations, wherein each of the plurality of inputs comprises a combination of a different respective one of feature subsets with the respective first latent representation.

### BACKGROUND

Neural networks are used in the field of machine learning and artificial intelligence (AI). A neural network comprises plurality of nodes which are interconnected by links, sometimes referred to as edges. The input edges of one or more nodes form the input of the network as a whole, and the output edges of one or more other nodes form the output of the network as a whole, whilst the output edges of various nodes within the network form the input edges to other nodes. Each node represents a function of its input edge(s) weighted by a respective weight, the result being output on its output edge(s). The weights can be gradually tuned based on a set of experience data (training data) so as to tend towards a state where the network will output a desired value for a given input.

Typically the nodes are arranged into layers with at least an input and an output layer. A “deep” neural network comprises one or more intermediate or “hidden” layers in between the input layer and the output layer. The neural network can take input data and propagate the input data through the layers of the network to generate output data. Certain nodes within the network perform operations on the data, and the result of those operations is passed to other nodes, and so on.

FIG. 1A gives a simplified representation of an example neural network 101 by way of illustration. The example neural network comprises multiple layers of nodes 104: an input layer 102i, one or more hidden layers 102h and an output layer 102o. In practice, there may be many nodes in each layer, but for simplicity only a few are illustrated. Each node 104 is configured to generate an output by carrying out a function on the values input to that node. The inputs to one or more nodes form the input of the neural network, the outputs of some nodes form the inputs to other nodes, and the outputs of one or more nodes form the output of the network.

At some or all of the nodes of the network, the input to that node is weighted by a respective weight. A weight may define the connectivity between a node in a given layer and the nodes in the next layer of the neural network. A weight can take the form of a single scalar value or can be modelled as a probabilistic distribution. When the weights are defined by a distribution, as in a Bayesian model, the neural network can be fully probabilistic and captures the concept of uncertainty. The values of the connections 106 between nodes may also be modelled as distributions. This is illustrated schematically in FIG. 1B. The distributions may be represented in the form of a set of samples or a set of parameters parameterizing the distribution (e.g. the mean μ and standard deviation a or variance σ²).

The network learns by operating on data input at the input layer, and adjusting the weights applied by some or all of the nodes based on the input data. There are different learning approaches, but in general there is a forward propagation through the network from left to right in FIG. 1A, a calculation of an overall error, and a backward propagation of the error through the network from right to left in FIG. 1A. In the next cycle, each node takes into account the back propagated error and produces a revised set of weights. In this way, the network can be trained to perform its desired operation.

The input to the network is typically a vector, each element of the vector representing a different corresponding feature. E.g. in the case of image recognition the elements of this feature vector may represent different pixel values, or in a medical application the different features may represent different symptoms or patient questionnaire responses. The output of the network may be a scalar or a vector. The output may represent a classification, e.g. an indication of whether a certain object such as an elephant is recognized in the image, or a diagnosis of the patient in the medical example.

FIG. 1C shows a simple arrangement in which a neural network is arranged to predict a classification based on an input feature vector. During a training phase, experience data comprising a large number of input data points X is supplied to the neural network, each data point comprising an example set of values for the feature vector, labelled with a respective corresponding value of the classification Y. The classification Y could be a single scalar value (e.g. representing elephant or not elephant), or a vector (e.g. a one-hot vector whose elements represent different possible classification results such as elephant, hippopotamus, rhinoceros, etc.). The possible classification values could be binary or could be soft-values representing a percentage probability. Over many example data points, the learning algorithm tunes the weights to reduce the overall error between the labelled classification and the classification predicted by the network. Once trained with a suitable number of data points, an unlabelled feature vector can then be input to the neural network, and the network can instead predict the value of the classification based on the input feature values and the tuned weights.

Training in this manner is sometimes referred to as a supervised approach. Other approaches are also possible, such as a reinforcement approach wherein the network each data point is not initially labelled. The learning algorithm begins by guessing the corresponding output for each point, and is then told whether it was correct, gradually tuning the weights with each such piece of feedback. Another example is an unsupervised approach where input data points are not labelled at all and the learning algorithm is instead left to infer its own structure in the experience data. The term “training” herein does not necessarily limit to a supervised, reinforcement or unsupervised approach.

A machine learning model (also known as a “knowledge model”) can also be formed from more than one constituent neural network. An example of this is an auto encoder, as illustrated by way of example in FIGS. 4A-D. In an auto encoder, an encoder network is arranged to encode an observed input vector Xₒ into a latent vector Z, and a decoder network is arranged to decode the latent vector back into the real-world feature space of the input vector. The difference between the actual input vector Xₒ and the version of the input vector X predicted by the decoder is used to tune the weights of the encoder and decoder so as to minimize a measure of overall difference, e.g. based on an evidence lower bound (ELBO) function. The latent vector Z can be thought of as a compressed form of the information in the input feature space. In a variational auto encoder (VAE), each element of the latent vector Z is modelled as a probabilistic or statistical distribution such as a Gaussian. In this case, for each element of Z the encoder learns one or more parameters of the distribution, e.g. a measure of centre point and spread of the distribution. For instance the centre point could be the mean and the spread could be the variance or standard deviation. The value of the element input to the decoder is then randomly sampled from the learned distribution.

The encoder is sometimes referred to as an inference network in that it infers the latent vector Z from an input observation Xₒ. The decoder is sometimes referred to as a generative network in that it generates a version \hat{X} of the input feature space from the latent vector Z.

Once trained, the auto encoder can be used to impute missing values from a subsequently observed feature vector Xₒ. Alternatively or additionally, a third network can be trained to predict a classification Y from the latent vector, and then once trained, used to predict the classification of a subsequent, unlabelled observation.

### SUMMARY

It is identified herein that conventional VAEs perform particularly poorly when the feature space of the input vector comprises mixed types of data. For example, in a medical setting, one or more of the features in the input feature space may be categorical values (e.g. a yes/no answer to a questionnaire, or gender) whilst one or more others may be continuous numerical values (e.g. height, or weight). Contrast for example with the case of image recognition where all the input features may represent pixel values.

In a VAE, the performance of any imputation or prediction performed based on the latent vector depends on the dimensionality of the latent space. In other words, the more elements (greater number of dimensions) are included in the latent vector, then the better the performance (where performance may be measured in terms of accuracy of prediction compared to a known ground truth in some test data). However, it is identified herein that when it comes to modelling mixed type data, the limiting factor on a conventional VAE is not the size of latent vector, but rather the mixed nature of the data types. It is identified herein that in such cases, increasing the latent size will not improve the performance significantly. On the other hand, the computational complexity (in terms of both training and prediction or imputation) will continue to scale with the dimensionality of the latent space (the number of elements in the latent vector Z) even if increasing the dimensionality is no longer increasing performance. Hence in applications where handling mixed types of data, conventional VAEs are not making efficient use of the computational complexity incurred.

It would be desirable to provide a machine learning model which can handle mixed types of data with reduced computational complexity for a given performance, or improved performance for a given computational complexity.

According to one aspect disclosed herein, there is provided a method comprising a first and a second stage. In the first stage, the method comprises training each of a plurality of individual first variational auto encoders, VAEs, each comprising an individual respective first encoder arranged to encode a respective subset of one or more features of a feature space into an individual respective first latent representation having one or more dimensions, and an individual respective first decoder arranged to decode from the respective latent representation back to a decoded version of the respective subset of the feature space, wherein different subsets comprise features of different types of data. In the second stage, following the first stage, the method comprises training a second VAE comprising a second encoder arranged to encode a plurality of inputs into a second latent representation having a plurality of dimensions, and a second decoder arranged to decode the second latent representation into decoded versions of the first latent representations, wherein each respective one of the plurality of inputs comprises a combination of a different respective one of feature subsets with the respective first latent representation.

As the first decoders are trained individually, separately from one another, then they can be trained without influencing one another. A second encoder and decoder can then be trained in a subsequent stage to encode into a second latent space and decode back to the individual first latent values, and thus learn the dependencies between the different data types. This two-stage approach, including a stage of separation between the different types of data, provides improved performance when handling mixed data.

In a conventional (“vanilla”) VAE, the dimensionality of the latent space is simply the dimensionality of the single latent vector Z between encoder and decoder. In the presently disclosed approach, the dimensionality is the sum of the dimensionality of the second latent representation (the number of elements in the second latent vector) plus the dimensionalities of each of the first latent representation (in embodiments one element each). E.g. the dimensionality may be represented as dim(H)+D, where dim(H) is the number of elements in the second latent vector H, and D is the number of features or feature subsets. However, an issue with a vanilla VAE is that under mixed type data, it cannot make use of the latent space very efficiently. Hence increasing the size of latent space will not help. On the contrary, since the disclosed method has a two-stage structure, it will actually have a larger latent size if H has the same dimensionality as Z. However, by disentangling the different feature types in the first learning stage, the increase of latent size in the disclosed model gives a significant boost compared with a vanilla VAE. So the latent space and training procedure are designed to make use of the latent space much more efficiently.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

### BRIEF DESCRIPTION OF THE DRAWINGS

To assist understanding of embodiments of the present disclosure and to show how such embodiments may be put into effect, reference is made, by way of example only, to the accompanying drawings in which:

FIG. 1A is a schematic illustration of a neural network,

FIG. 1B is a schematic illustration of a node of a Bayesian neural network,

FIG. 1C is a schematic illustration of a neural network arranged to predict a classification based on an input feature vector,

FIG. 2 is a schematic illustration of a computing apparatus for implementing a neural network,

FIG. 3 schematically illustrates a data set comprising a plurality of data points each comprising one or more feature values,

FIG. 4A is a schematic illustration of a variational auto encoder (VAE),

FIG. 4B is another schematic representation of a VAE,

FIG. 4C is a high-level schematic representation of a VAE,

FIG. 4D is a high-level schematic representation of a VAE,

FIG. 5A schematically illustrates a first stage of training a machine learning model in accordance with embodiments disclosed herein,

FIG. 5B is a schematically illustrates a second stage of training a machine learning model in accordance with embodiments disclosed herein,

FIG. 5C is a high-level schematic representation of the knowledge model of FIGS. 5A and 5B,

FIG. 5D illustrates a variant of the decoder in the model of FIGS. 5A and 5B,

FIG. 5E illustrates use of the model to predict a classification,

FIG. 6 illustrates a partial inference network for imputing missing values,

FIG. 7A shows pair plots of 3-dimensional data for a ground truth,

FIG. 7B shows pair plots of 3-dimensional data generated using a model,

FIG. 7C shows pair plots of 3-dimensional data generated using another model,

FIG. 7D shows pair plots of 3-dimensional data generated using another model,

FIG. 7E shows pair plots of 3-dimensional data generated using another model,

FIG. 7F shows pair plots of 3-dimensional data generated using another model,

FIG. 8 shows (a)-(e) some information curves of sequential active information acquisition for some example scenarios and (f) a corresponding area under information curve (AUIC) comparison, and

FIG. 9 is a flow chart of an overall method in accordance with the presently disclosed techniques.

### DETAILED DESCRIPTION OF EMBODIMENTS

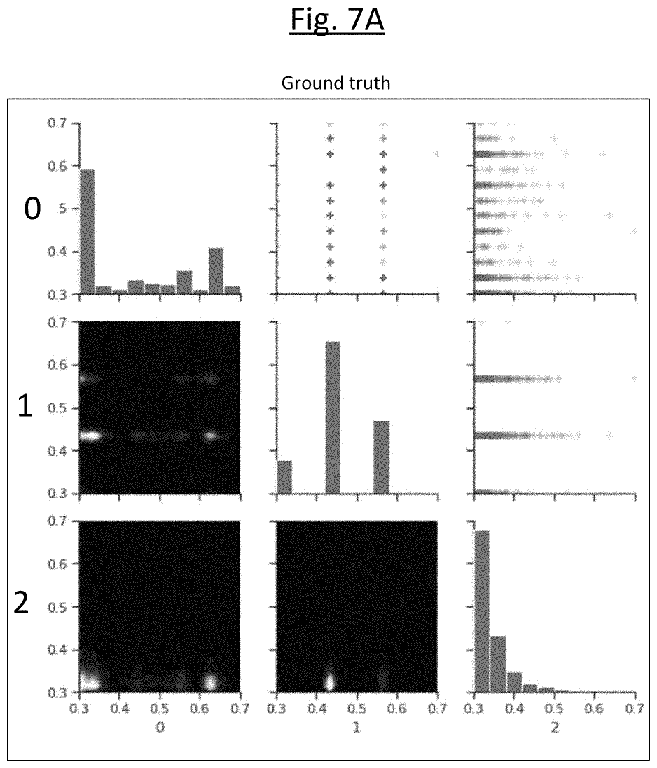

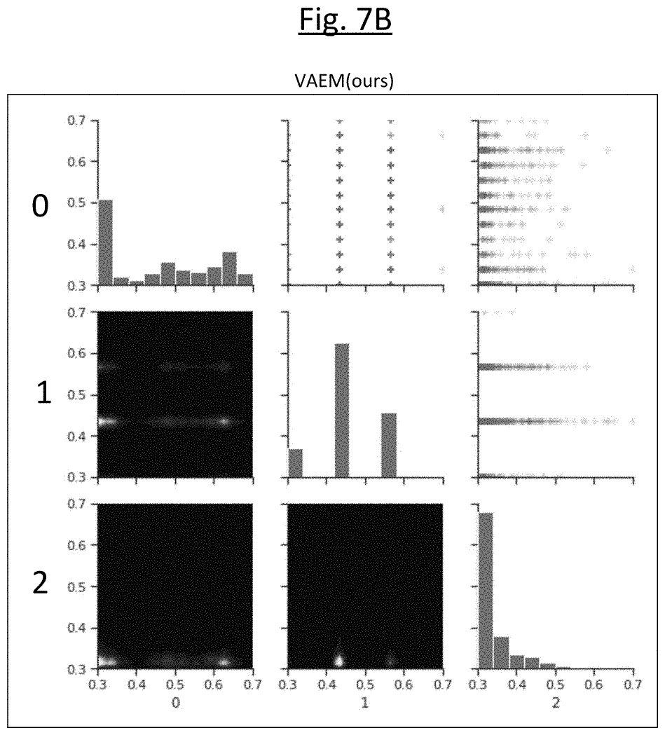

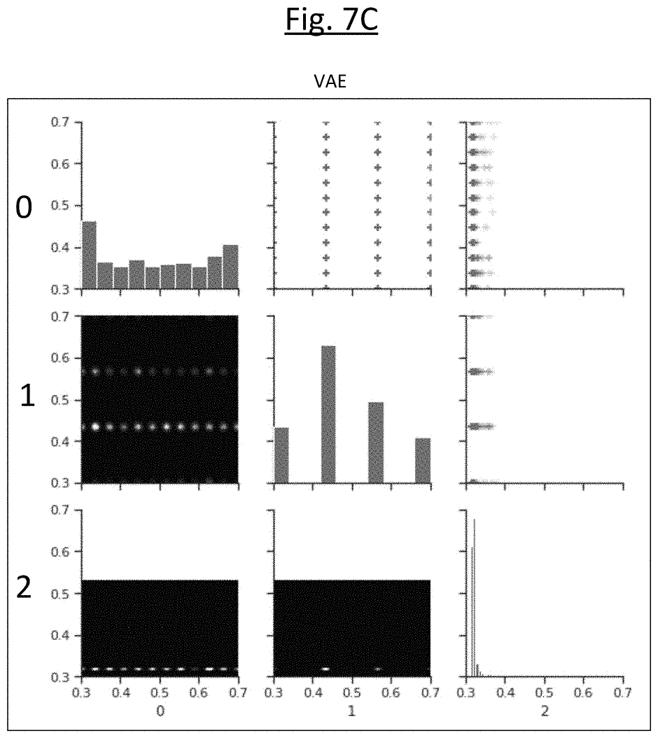

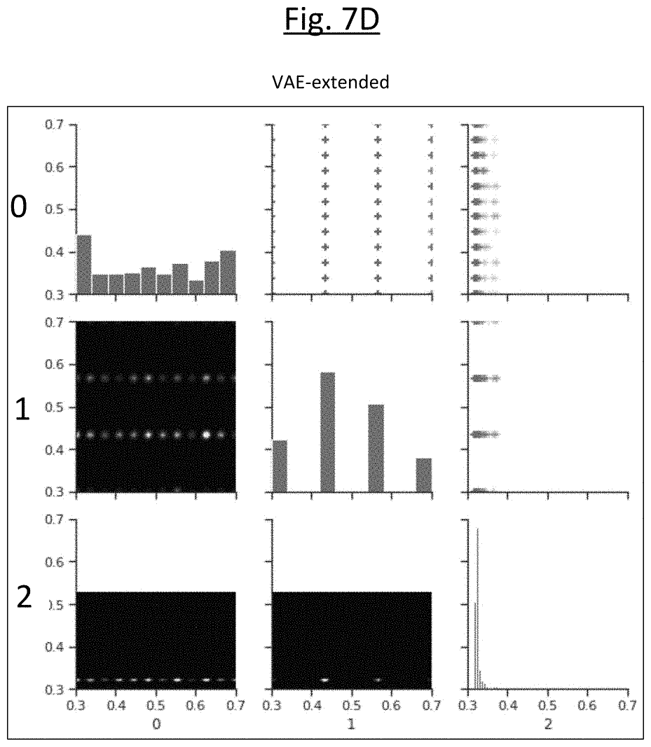

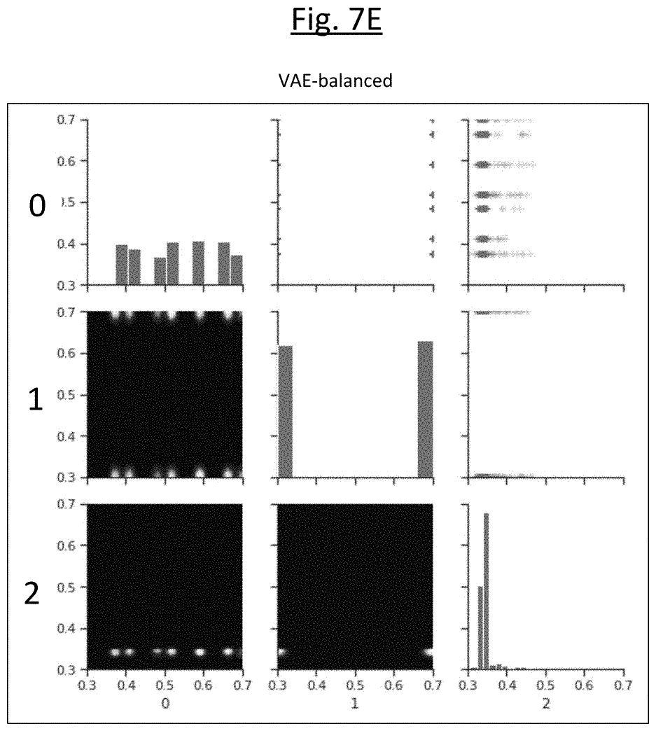

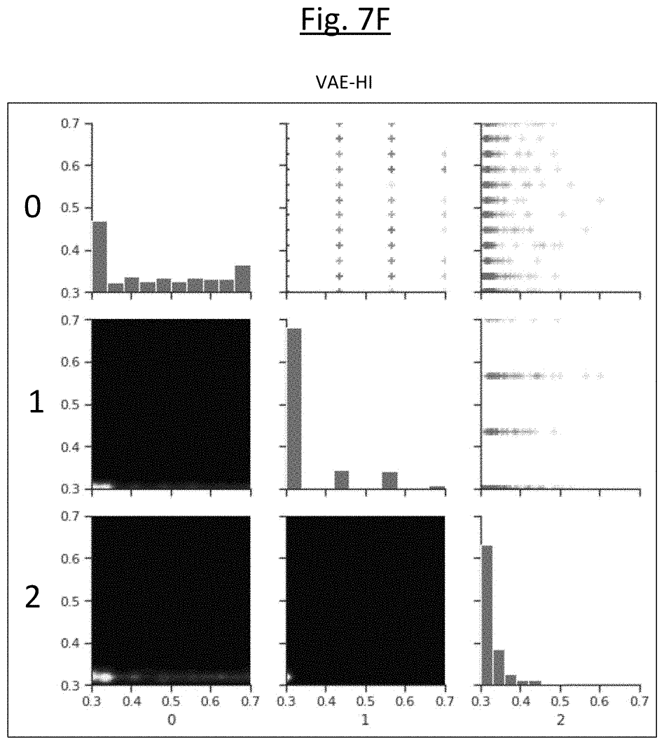

Deep generative models often perform poorly in real-world applications due to the heterogeneity of natural data sets. Heterogeneity arises from having different types of features (e.g. categorical, continuous, etc.) each with their own marginal properties which can be drastically different. “Marginal” refers to the distribution of different possible values of the feature verses number of samples, disregarding co-dependency with other features. In other words the shape of the distribution for different types of feature can be quite different. The types of data may include for example: categorical (the value of the feature takes one of a plurality of non-numerical categories), ordinal (integer numerical values) and/or continuous (continuous numerical values). VAE will try to optimize all likelihood functions all at once. In practice some likelihood functions may have larger values, hence the VAE will pay attention to a particular likelihood function and ignore others. In this case, the contribution that each likelihood makes to the training objective can be very different, leading to challenging optimization problems in which some data dimensions may be poorly-modelled in favour of others. FIG. 7 (d) shows an example in which a vanilla VAE fits some of the categorical variables but performs poorly on the continuous ones.

Using VAEs for modelling mixed type real-world data is under explored in the literature, especially when combined with down-stream decision-making tasks. To overcome the limitations of VAEs in this setting, the present disclosure provides a new method which may be referred to as a variational auto-encoder for heterogeneous mixed type data (VAEM). Later some examples of its performance for decision making in real-world applications are studied. VAEM uses a hierarchy of latent variables which is fit in two stages. In the first stage, one type-specific VAE is learned for each dimension. These initial one-dimensional VAEs capture marginal distribution properties and provide a latent representation that is uniform across dimensions. In the second stage, another VAE is used to capture dependencies among the one-dimensional latent representations from the first stage.

Thus there is provided an improved model for heterogeneous mixed type data which alleviates the limitations of conventional VAEs. In embodiments the VAEM employs a deep generative model for the heterogeneous mixed type data.

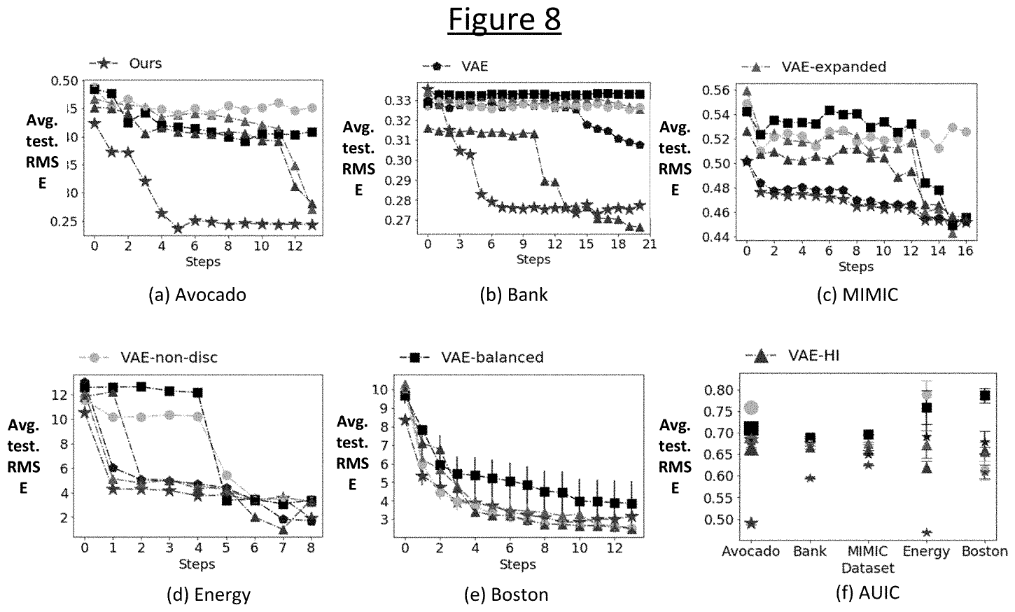

The disclosure herein will study the data generation quality of VAEM comparing with VAEs and other baselines on five different datasets (e.g. see FIG. 8). The results show that VAEM can model mixed type data more successfully than other baselines.

In embodiments, VAEM may be extended to handle missing data, perform conditional data generation, and employ algorithms that enable it to be used for efficient sequential active information acquisition. It will be shown herein that VAEM obtains strong performance for conditional data generation as well as sequential active information acquisition in cases where VAEs perform poorly.

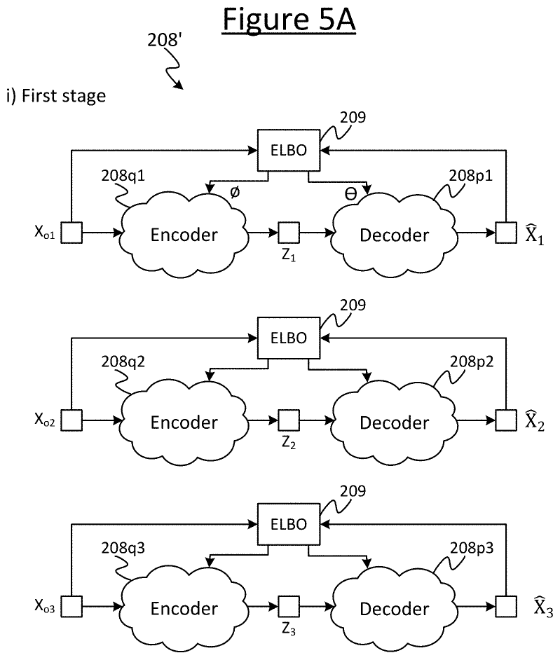

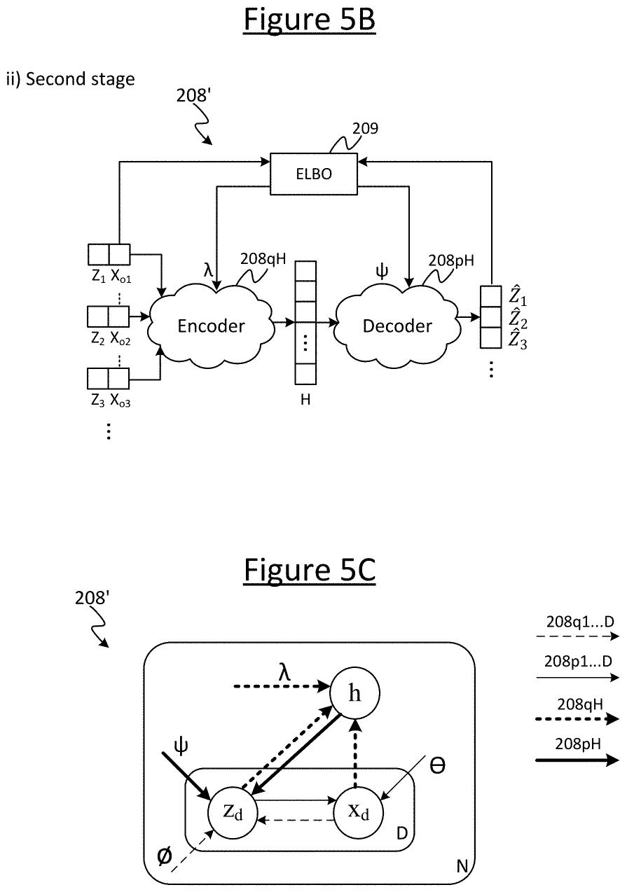

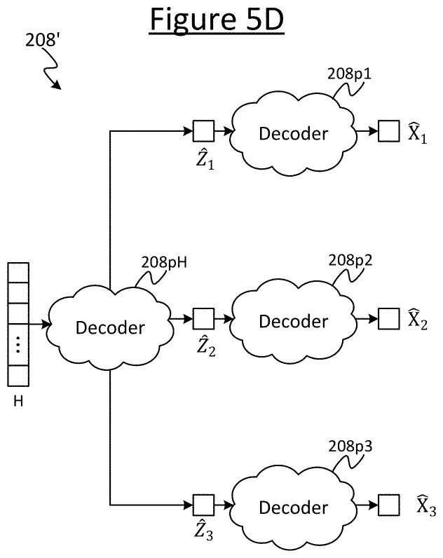

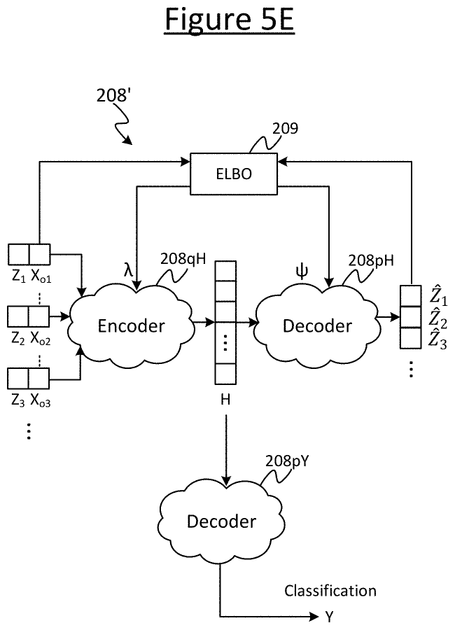

The two-stage VAEM model will be discussed in more detail shortly with reference to FIG. 5A onwards. First however a general overview of neural networks and their use in VAEs will is discussed with reference to FIGS. 2 to 4D.

FIG. 2 illustrates an example computing apparatus 200 for implementing an artificial intelligence (AI) algorithm including a machine-learning (ML) model in accordance with embodiments described herein. The computing apparatus 200 may comprise one or more user terminals, such as a desktop computer, laptop computer, tablet, smartphone, wearable smart device such as a smart watch, or an on-board computer of a vehicle such as car, etc. Additionally or alternatively, the computing apparatus 200 may comprise a server. A server herein refers to a logical entity which may comprise one or more physical server units located at one or more geographic sites. Where required, distributed or “cloud” computing techniques are in themselves known in the art. The one or more user terminals and/or the one or more server units of the server may be connected to one another via a packet-switched network, which may comprise for example a wide-area internetwork such as the Internet, a mobile cellular network such as a 3GPP network, a wired local area network (LAN) such as an Ethernet network, or a wireless LAN such as a Wi-Fi, Thread or 6LoWPAN network.

The computing apparatus 200 comprises a controller 202, an interface 204, and an artificial intelligence (AI) algorithm 206. The controller 202 is operatively coupled to each of the interface 204 and the AI algorithm 206.

Each of the controller 202, interface 204 and AI algorithm 206 may be implemented in the form of software code embodied on computer readable storage and run on processing apparatus comprising one or more processors such as CPUs, work accelerator co-processors such as GPUs, and/or other application specific processors, implemented on one or more computer terminals or units at one or more geographic sites. The storage on which the code is stored may comprise one or more memory devices employing one or more memory media (e.g. electronic or magnetic media), again implemented on one or more computer terminals or units at one or more geographic sites. In embodiments, one, some or all the controller 202, interface 204 and AI algorithm 206 may be implemented on the server. Alternatively, a respective instance of one, some or all of these components may be implemented in part or even wholly on each of one, some or all of the one or more user terminals. In further examples, the functionality of the above-mentioned components may be split between any combination of the user terminals and the server. Again it is noted that, where required, distributed computing techniques are in themselves known in the art. It is also not excluded that one or more of these components may be implemented in dedicated hardware.

The controller 202 comprises a control function for coordinating the functionality of the interface 204 and the AI algorithm 206. The interface 204 refers to the functionality for receiving and/or outputting data. The interface 204 may comprise a user interface (UI) for receiving and/or outputting data to and/or from one or more users, respectively; or it may comprise an interface to one or more other, external devices which may provide an interface to one or more users. Alternatively the interface may be arranged to collect data from and/or output data to an automated function or equipment implemented on the same apparatus and/or one or more external devices, e.g. from sensor devices such as industrial sensor devices or IoT devices. In the case of interfacing to an external device, the interface 204 may comprise a wired or wireless interface for communicating, via a wired or wireless connection respectively, with the external device. The interface 204 may comprise one or more constituent types of interface, such as voice interface, and/or a graphical user interface.

The interface 204 is thus arranged to gather observations (i.e. observed values) of various features of an input feature space. It may for example be arranged to collect inputs entered by one or more users via a UI front end, e.g. microphone, touch screen, etc.; or to automatically collect data from unmanned devices such as sensor devices. The logic of the interface may be implemented on a server, and arranged to collect data from one or more external user devices such as user devices or sensor devices. Alternatively some or all of the logic of the interface 204 may also be implemented on the user device(s) or sensor devices its/themselves.

The controller 202 is configured to control the AI algorithm 206 to perform operations in accordance with the embodiments described herein. It will be understood that any of the operations disclosed herein may be performed by the AI algorithm 206, under control of the controller 202 to collect experience data from the user and/or an automated process via the interface 204, pass it to the AI algorithm 206, receive predictions back from the AI algorithm and output the predictions to the user and/or automated process through the interface 204.

The machine learning (ML) algorithm 206 comprises a machine-learning model 208, comprising one or more constituent neural networks 101. A machine-leaning model 208 such as this may also be referred to as a knowledge model. The machine learning algorithm 206 also comprises a learning function 209 arranged to tune the weights w of the nodes 104 of the neural network(s) 101 of the machine-learning model 208 according to a learning process, e.g. training based on a set of training data.

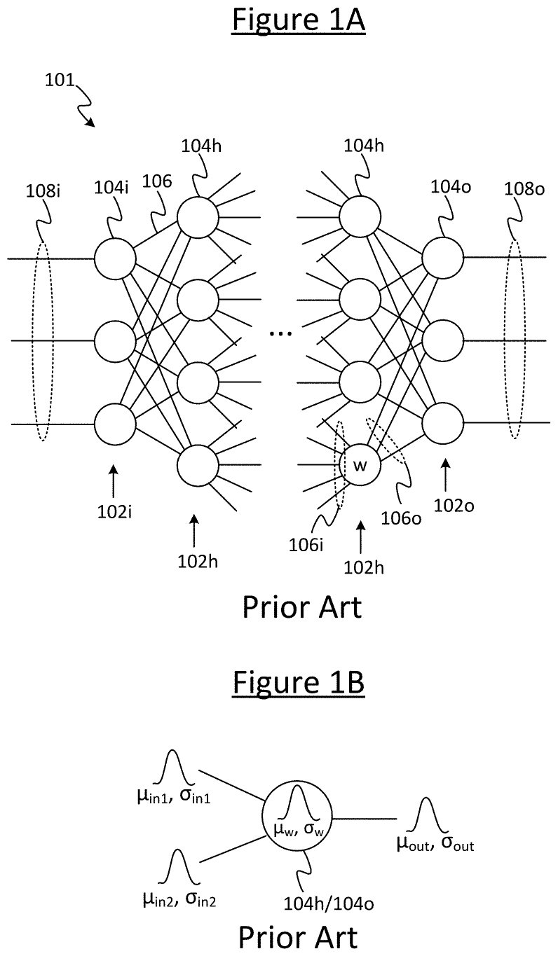

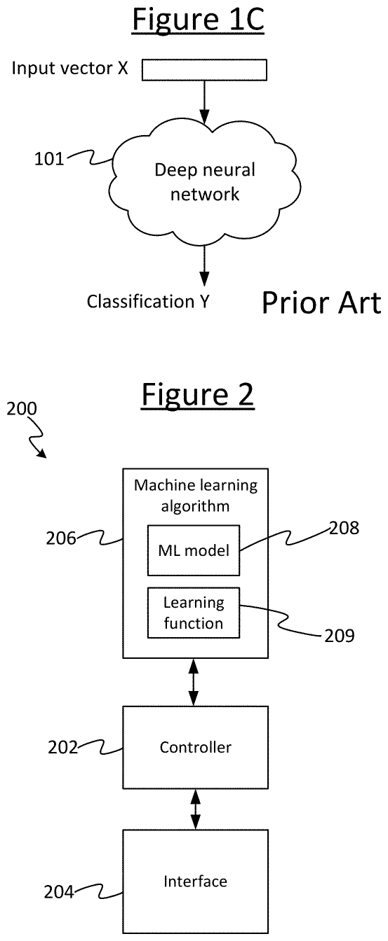

FIG. 1A illustrates the principle behind a neural network. A neural network 101 comprises a graph of interconnected nodes 104 and edges 106 connecting between nodes, all implemented in software. Each node 104 has one or more input edges and one or more output edges, with at least some of the nodes 104 having multiple input edges per node, and at least some of the nodes 104 having multiple output edges per node. The input edges of one or more of the nodes 104 form the overall input 108i to the graph (typically an input vector, i.e. there are multiple input edges). The output edges of one or more of the nodes 104 form the overall output 108o of the graph (which may be an output vector in the case where there are multiple output edges). Further, the output edges of at least some of the nodes 104 form the input edges of at least some others of the nodes 104.

Each node 104 represents a function of the input value(s) received on its input edges(s) 106i, the outputs of the function being output on the output edge(s) 106o of the respective node 104, such that the value(s) output on the output edge(s) 106o of the node 104 depend on the respective input value(s) according to the respective function. The function of each node 104 is also parametrized by one or more respective parameters w, sometimes also referred to as weights (not necessarily weights in the sense of multiplicative weights, though that is certainly one possibility). Thus the relation between the values of the input(s) 106i and the output(s) 106o of each node 104 depends on the respective function of the node and its respective weight(s).

Each weight could simply be a scalar value. Alternatively, as shown in FIG. 1B, at some or all of the nodes 104 in the network 101, the respective weight may be modelled as a probabilistic distribution such as a Gaussian. In such cases the neural network 101 is sometimes referred to as a Bayesian neural network. Optionally, the value input/output on each of some or all of the edges 106 may each also be modelled as a respective probabilistic distribution. For any given weight or edge, the distribution may be modelled in terms of a set of samples of the distribution, or a set of parameters parameterizing the respective distribution, e.g. a pair of parameters specifying its centre point and width (e.g. in terms of its mean μ and standard deviation a or variance σ²). The value of the edge or weight may be a random sample from the distribution. The learning or the weights may comprise tuning one or more of the parameters of each distribution.

As shown in FIG. 1A, the nodes 104 of the neural network 101 may be arranged into a plurality of layers, each layer comprising one or more nodes 104. In a so-called “deep” neural network, the neural network 101 comprises an input layer 102i comprising one or more input nodes 104i, one or more hidden layers 102h (also referred to as inner layers) each comprising one or more hidden nodes 104h (or inner nodes), and an output layer 102o comprising one or more output nodes 104o. For simplicity, only two hidden layers 102h are shown in FIG. 1A, but many more may be present.

The different weights of the various nodes 104 in the neural network 101 can be gradually tuned based on a set of experience data (training data), so as to tend towards a state where the output 108o of the network will produce a desired value for a given input 108i. For instance, before being used in an actual application, the neural network 101 may first be trained for that application. Training comprises inputting experience data in the form of training data to the inputs 108i of the graph and then tuning the weights w of the nodes 104 based on feedback from the output(s) 108o of the graph. The training data comprises multiple different input data points, each comprising a value or vector of values corresponding to the input edge or edges 108i of the graph 101.

For instance, consider a simple example as in FIG. 1C where the machine-learning model comprises a single neural network 101, arranged to take a feature vector X as its input 108i and to output a classification Y as its output 108o. The input feature vector X comprises a plurality of elements x_{d}, each representing a different feature d=0, 1, 2, . . . etc. E.g. in the example of image recognition, each element of the feature vector X may represent a respective pixel value. For instance one element represents the red channel for pixel (0,0); another element represents the green channel for pixel (0,0); another element represents the blue channel of pixel (0,0); another element represents the red channel of pixel (0,1); and so forth. As another example, where the neural network is used to make a medical diagnosis, each of the elements of the feature vector may represent a value of a different symptom of the subject, physical feature of the subject, or other fact about the subject (e.g. body temperature, blood pressure, etc.).

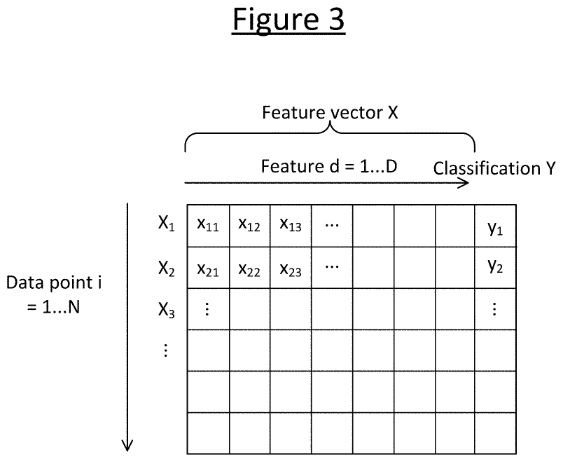

FIG. 3 shows an example data set comprising a plurality of data points i=0, 1, 2, . . . etc. Each data point i comprises a respective set of values of the feature vector (where x_{id} is the value of the dₜₕ feature in the iₜₕ data point). The input feature vector Xᵢ represents the input observations for a given data point, where in general any given observation i may or may not comprise a complete set of values for all the elements of the feature vector X. The classification Yᵢ represents a corresponding classification of the observation i. In the training data an observed value of classification Yᵢ is specified with each data point along with the observed values of the feature vector elements (the input data points in the training data are said to be “labelled” with the classification Yᵢ). In subsequent a prediction phase, the classification Y is predicted by the neural network 101 for a further input observation X.

The classification Y could be a scalar or a vector. For instance in the simple example of the elephant-recognizer, Y could be a single binary value representing either elephant or not elephant, or a soft value representing a probability or confidence that the image comprises an image of an elephant. Or similarly, if the neural network 101 is being used to test for a particular medical condition, Y could be a single binary value representing whether the subject has the condition or not, or a soft value representing a probability or confidence that the subject has the condition in question. As another example, Y could comprise a “1-hot” vector, where each element represents a different animal or condition. E.g. Y=[1, 0, 0, . . . ] represents an elephant, Y=[0, 1, 0, . . . ] represents an hippopotamus, Y=[0, 0, 1, . . . ] represents a rhinoceros, etc. Or if soft values are used, Y=[0.81, 0.12, 0.05, . . . ] represents an 81% confidence that the image comprises an image of an elephant, 12% confidence that it comprises an image of an hippopotamus, 5% confidence of a rhinoceros, etc.

In the training phase, the true value of Yᵢ for each data point i is known. With each training data point i, the AI algorithm 206 measures the resulting output value(s) at the output edge or edges 108o of the graph, and uses this feedback to gradually tune the different weights w of the various nodes 104 so that, over many observed data points, the weights tend towards values which make the output(s) 108i (Y) of the graph 101 as close as possible to the actual observed value(s) in the experience data across the training inputs (for some measure of overall error). I.e. with each piece of input training data, the predetermined training output is compared with the actual observed output of the graph 108o. This comparison provides the feedback which, over many pieces of training data, is used to gradually tune the weights of the various nodes 104 in the graph toward a state whereby the actual output 108o of the graph will closely match the desired or expected output for a given input 108i. Examples of such feedback techniques include for instance stochastic back-propagation.

Once trained, the neural network 101 can then be used to infer a value of the output 108o (Y) for a given value of the input vector 108i (X), or vice versa.

Explicit training based on labelled training data is sometimes referred to as a supervised approach. Other approaches to machine learning are also possible. For instance another example is the reinforcement approach. In this case, the neural network 101 begins making predictions of the classification Yᵢ for each data point i, at first with little or no accuracy. After making the prediction for each data point i (or at least some of them), the AI algorithm 206 receives feedback (e.g. from a human) as to whether the prediction was correct, and uses this to tune the weights so as to perform better next time. Another example is referred to as the unsupervised approach. In this case the AI algorithm receives no labelling or feedback and instead is left to infer its own structure in the experienced input data.

FIG. 1C is a simple example of the use of a neural network 101. In some cases, the machine-learning model 208 may comprise a structure of two or more constituent neural networks 101.

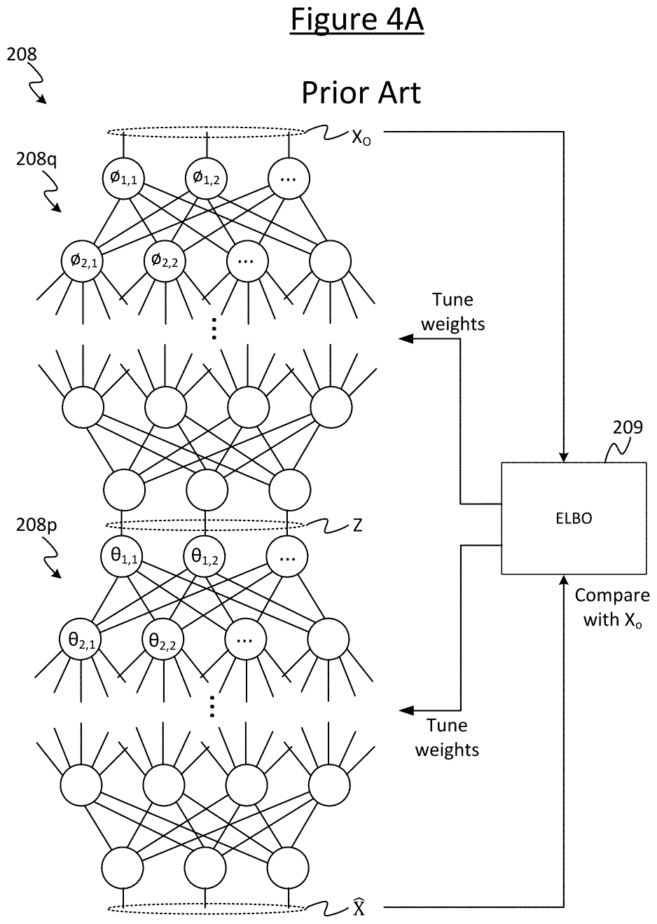

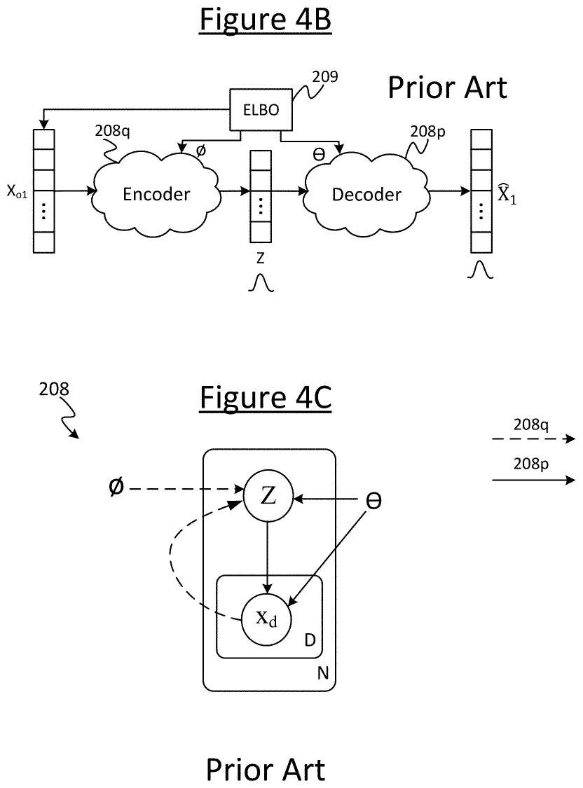

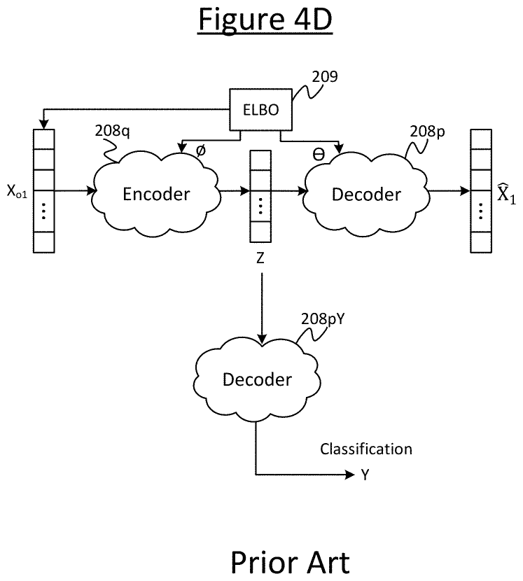

FIG. 4A schematically illustrates one such example, known as a variational auto encoder (VAE). In this case the machine learning model 208 comprises an encoder 208q comprising an inference network, and a decoder 208p comprising a generative network. Each of the inference networks and the generative networks comprises one or more constituent neural networks 101, such as discussed in relation to FIG. 1A. An inference network for the present purposes means a neural network arranged to encode an input into a latent representation of that input, and a generative network means a neural network arranged to at least partially decode from a latent representation.

The encoder 208q is arranged to receive the observed feature vector Xₒ as an input and encode it into a latent vector Z (a representation in a latent space). The decoder 208p is arranged to receive the latent vector Z and decode back to the original feature space of the feature vector. The version of the feature vector output by the decoder 208p may be labelled herein \hat{X}.

The latent vector Z is a compressed (i.e. encoded) representation of the information contained in the input observations Xₒ. No one element of the latent vector Z necessarily represents directly any real world quantity, but the vector Z as a whole represents the information in the input data in compressed form. It could be considered conceptually to represent abstract features abstracted from the input data Xₒ, such as “wrinklyness” and “trunk-like-ness” in the example of elephant recognition (though no one element of the latent vector Z can necessarily be mapped onto any one such factor, and rather the latent vector Z as a whole encodes such abstract information). The decoder 208p is arranged to decode the latent vector Z back into values in a real-world feature space, i.e. back to an uncompressed form \hat{X} representing the actual observed properties (e.g. pixel values). The decoded feature vector \hat{X} has the same number of elements representing the same respective features as the input vector Xₒ.

The weights w of the inference network (encoder) 208q are labelled herein ø, whilst the weights w of the generative network (decoder) 208p are labelled θ. Each node 104 applies its own respective weight as illustrated in FIG. 4.

With each data point in the training data (each data point in the experience data during learning), the learning function 209 tunes the weights ø and θ so that the VAE 208 learns to encode the feature vector X into the latent space Z and back again. For instance, this may be done by minimizing a measure of divergence between q_{ø}(Zᵢ|Xᵢ) and p_{θ}(Xᵢ|Zᵢ), where q_{ø}(Zᵢ|Xᵢ) is a function parameterised by ø representing a vector of the probabilistic distributions of the elements of Zᵢ output by the encoder 208q given the input values of Xᵢ, whilst p_{θ}(Xᵢ|Zᵢ) is a function parameterized by θ representing a vector of the probabilistic distributions of the elements of Xᵢ output by the encoder 208q given Zᵢ. The symbol “|” means “given”. The model is trained to reconstruct Xᵢ and therefore maintains a distribution over Xᵢ. At the “input side”, the value of Xoᵢ is known, and at the “output side”, the likelihood of \hat{X}ᵢ under the output distribution of the model is evaluated. Typically p(z|x) is referred to as posterior, and q(z|x) as approximate posterior. p(z) and q(z) are referred to as priors.

For instance, this may be done by minimizing the Kullback-Leibler (KL) divergence between q_{ø}(Zᵢ|Xᵢ) and p_{θ}(Xᵢ|Zᵢ). The minimization may be performed using an optimization function such as an ELBO (evidence lower bound) function, which uses cost function minimization based on gradient descent. An ELBO function may be referred to herein by way of example, but this is not limiting and other metrics and functions are also known in the art for tuning the encoder and decoder networks of a VAE.

The requirement to learn to encode to Z and back again amounts to a constraint placed on the overall neural network 208 of the VAE formed from the constituent neural networks of the encoder and decoder 208q, 208p. This is the general principle of an autoencoder. The purpose of forcing the autoencoder to learn to encode and then decode a compressed form of the data, is that this can achieve one or more advantages in the learning compared to a generic neural network; such as learning to ignore noise in the input data, making better generalizations, or because when far away from a solution the compressed form gives better gradient information about how to quickly converge to a solution. In a variational autoencoder, the latent vector Z is subject to an additional constraint that it follows a predetermined form of probabilistic distribution such as a multidimensional Gaussian distribution or gamma distribution.

FIG. 4B shows a more abstracted representation of a VAE such as shown in FIG. 4A.

FIG. 4C shows an even higher level representation of a VAE such as that shown in FIGS. 4A and 4B. In FIG. 4C the solid lines represent a generative network of the decoder 208q, and the dashed lines represents an inference network of the encoder 208p. In this form of diagram, a vector shown in a circle represents a vector of distributions. So here, each element of the feature vector X (=x₁ . . . x_{d}) is modelled as a distribution, e.g. as discussed in relation to FIG. 1C. Similarly each element of the latent vector Z is modelled as a distribution. On the other hand, a vector shown without a circle represents a fixed point. So in the illustrated example, the weights e of the generative network are modelled as simple values, not distributions (though that is a possibility as well). The rounded rectangle labelled N represents the “plate”, meaning the vectors within the plate are iterated over a number N of learning steps (one for each data point). In other words i=0, . . . , N−1. A vector outside the plate is global, i.e. it does not scale with the number of data points i (nor the number of features d in the feature vector). The rounded rectangle labelled D represents that the feature vector X comprises multiple elements x₁ . . . x_{d}.

There are a number of ways that a VAE 208 can be used for a practical purpose. One use is, once the VAE has been trained, to generate a new, unobserved instance of the feature vector \hat{X} by inputting a random or unobserved value of the latent vector Z into the decoder 208p. For example if the feature space of X represents the pixels of an image, and the VAE has been trained to encode and decode human faces, then by inputting a random value of Z into the decoder 208p it is possible to generate a new face that did not belong to any of the sampled subjects during training. E.g. this could be used to generate a fictional character for a movie or video game.

Another use is to impute missing values. In this case, once the VAE has been trained, another instance of an input vector Xₒ may be input to the encoder 208q with missing values. I.e. no observed value of one or more (but not all) of the elements of the feature vector Xₒ. The values of these elements (representing the unobserved features) may be set to zero, or 50%, or some other predetermined value representing “no observation.” The corresponding element(s) in the decoded version of the feature vector \hat{X} can then be read out from the decoder 208p in order to impute the missing value(s). The VAE may also be trained using some data points that have missing values of some features.

Another possible use of a VAE is to predict a classification, similarly to the idea described in relation to FIG. 1A. In this case, illustrated in FIG. 4D, a further decoder 208pY is arranged to decode the latent vector Z into a classification Y, which could be a single element or a vector comprising multiple elements (e.g. a one-hot vector). During training, each input data point (each observation of Xₒ) is labelled with an observed value of the classification Y, and the further decoder 208pY is thus trained to decode the latent vector Z into the classification Y. After training, this can then be used to input an unlabelled feature vector Xₒ and have the decoder 208pY generate a prediction of the classification Y for the observed feature vector Xₒ.

An improved method of forming a machine learning model 208′, in accordance with embodiments disclosed herein, is now described with reference to FIGS. 5A-5E. Particularly, the method disclosed herein is particularly suited to handling mixed types of data. This machine learning (ML) model 208′ can be used in place of a standard VAE in the apparatus 200 of FIG. 2, for example, in order to make predictions or perform imputations.

The model is trained in two stages. In a first stage, an individual VAE is trained for each of the individual features or feature types, without one influencing another. In a second stage, a further VAE is then trained to learn the inter-feature dependencies.

Both a vanilla VAE and the disclosed form of VAE use multiple likelihood functions. However, the issue with a vanilla VAE is that it tries to optimize all likelihood functions all at once. In practice some likelihood functions may have larger values, hence the VAE will pay attention to a particular likelihood function and ignore others. In contrast, the disclosed method works to optimize all likelihood function separately so that it mitigates this issue.

As shown in FIG. 5A, in the first stage an individual VAE is trained for each of multiple different subsets Xₒ₁, Xₒ₂, Xₒ₃ of features of the observed feature vector Xₒ (i.e. different subsets of the feature space of the vector). Three subsets are shown here by way of illustration, but it will be appreciated that other numbers could be used. Each subset comprises a different respective one or more of the features of the feature space. I.e. each subset is a different one or more of the elements of the feature vector Xₒ. The different subsets of features comprise features of different data types. For instance, the types may comprise two or more of: categorical, ordinal, or continuous. Categorical refers to data whose value takes one of a discrete number of categories. An example of this could be gender, or a response to a question with a discrete number of qualitative answers. In some cases categorical data could be divided into two types: binary categorical and non-binary categorical. E.g. an example of binary data would be answers to a yes/no question, or smoker/non-smoker. An example of non-binary data could be gender, e.g. male, female or other; or town or country of residence, etc. An example of ordinal data would be age measured in completed years, or a response to a question giving a ranking on a scale of 1 to 10, or one or five stars, or such like. An example of continuous data would be weight or height. It will be appreciated that these different types of data have very different statistical properties.

The number of subsets may be labelled herein d=1 . . . D, where d is an index of the subset and D is the total number of subsets. In embodiments, each subset X_{od} is only a single respective feature. E.g. one feature Xₒ₁ could be gender, another feature Xₒ₂ could be age, whilst another feature Xₒ₃ could be weight (such as in an example for predicting or imputing a medical condition of a user). Alternatively features of the same type could be grouped together into the subset trained by one of the individual VAEs. E.g. one subset Xₒ₁ could consist of categorical variables, another subset Xₒ₂ could consist of ordinal variables, whilst another subset Xₒ₃ could consist of continuous variables.

Each individual VAE comprises a respective first encoder 208q_{d} (d=1 . . . D) arranged to encode the respective feature X_{od} into a respective latent representation (i.e. latent space) Z_{d}. Each individual VAE also comprises a respective first decoder 208p_{d} (d=1 . . . D) arranged to decode the respective latent representation Z_{d} back into the respective dimension(s) of the feature space of the respective subset of features, i.e. to generate a decoded version \hat{X}_{d} of the respective observed feature subset X_{od}. So Xₒ₁ is encoded into Z₁ and then decoded into \hat{X}₁, whilst Xₒ₂ _{²} is encoded into Z₂ and then decoded into \hat{X}₂, and Xₒ₃ is encoded into Z₃ and then decoded into \hat{X}₃ (and so forth if there are more than three feature subsets.

In embodiments each of the latent representations Z_{d} is one-dimensional, i.e. consists of only a single latent variable (element). Note however this does not imply the latent variable Z_{d} is a modelled only as simple, fixed scalar value. Rather, as the auto-encoder is a variation auto-encoder, then for each latent variable Z_{d} the encoder learns a statistical or probabilistic distribution, and the value input to the decoder is a random sample from the distribution. This means that for each individual element of latent space, the encoder learns one or more parameters of the respective distribution, e.g. a measure of centre point and spread of the distribution. For instance each latent variable Z_{d} (a single dimension) may be modelled in the encoder by a respective mean value μ_{d} and standard deviation σ_{d} or variance σ_{d}². The possibility of a multi-dimensional Z_{d} is also not excluded (in which case each dimension is modelled by one or more parameters of a respective distribution), though this would increase computational complexity, and generally the idea of a latent representation is that it compresses the information from the input feature space into a lower dimensionality.

In the first stage, each individual VAE is trained (i.e. has it weights tuned) by the learning function 209 (e.g. an ELBO function) to minimize a measure of difference between the respective observed feature subset X_{od} and the respective decoded version of that feature subset \hat{X}_{d}.

As shown in FIG. 5B, the second stage employs a second VAE comprising a second decoder 208pH and second encoder 208qH to form a second VAE. The second stage of the method comprises training this second VAE.

At the input of the second encoder 208qH, each of the feature subsets X_{od} is combined with its respective latent vector Z_{d} (using the values of Z_{d} learned using the first VAE in the first stage). In embodiments this combination comprises concatenating each feature subset X_{od} with its respective latent vector Z_{d}. However in principle any function which combines the information of the two could be used, e.g. a multiplication, or interleaving, etc. Whatever function is used, each such combination forms one of the inputs of the second encoder 208qH. The second encoder 208qH is arranged to encode these inputs into a second latent representation in the form of a latent vector H, having multiple dimensions (with each dimension—i.e. each element of the vector—being modelled as a respective distribution, so represented in terms of one or more parameters of the respective distribution, e.g. a respective mean and variance or standard deviation). H is also referred to later as h (in the vector form), not to be confused with h(⋅) the function.

The second decoder 208pH is arranged to decode the second latent representation H back into a version of the individual first latent representations \hat{Z}_{d} (d=1 . . . D). In the second learning stage, the second VAE is trained (i.e. has its weights tuned) by the learning function 209 to minimize a measure of difference between the first latent representation Z and the decoded version thereof \hat{Z} (where Z is the vector made up of the individual first latent representations Z₁, Z₂, Z₃, . . . ; and \hat{Z}₁, \hat{Z}₂, \hat{Z}₃, . . . are the corresponding decoded versions). In FIGS. 5A-5B the weights of the first encoder 208qd are represented by Ø, the weights of the first decoder 208pd are represented by θ, the weights of the second encoder 208qh are represented by λ, and the weights of the second decoder 208pH are represented by ψ.

A more abstracted, higher-level representation of the model 208′ is shown in FIG. 5C.

Based on this two-stage approach, the model thus learns to first disentangle the dependencies between different data types, and then learn to the effect of the dependencies between data types.

The computational complexity of an auto-encoder increases with the dimensionality of the latent space. For instance, consider a conventional VAE 208 as shown in FIGS. 4A-D, which encodes into a latent vector Z having 20 elements (each modelled as a distribution). The dimensionality of this is said to be 20. Now consider an implementation of the presently disclosed model 208′ where H has 20 elements, each Z_{d} is a single element and D=3. The total overall dimensionality of the latent space in this case is 20+1+1+1=23. Or more generally dim(H)+D where dim(H) is the number of dimensions (elements) of H and D is the number of features or feature subsets (i.e. the number of first encoder/decoders). However, an issue with a vanilla VAE is that under mixed type data, it cannot make use of the latent space very efficiently. Hence increasing the size of latent space will not help. On the contrary, by disentangling the different feature types in the first learning stage, then in the presently disclosed approach the increase of latent size in the disclosed model improves performance compared with the vanilla VAE. So the latent space and training procedure are designed to make use of the latent space much more efficiently.

As shown in FIG. 5D, once trained the model 208′ can be used to make predictions or perform imputations in an analogous manner to that described in relation to FIGS. 4A-4D. A value of the second latent vector H is input to the second decoder which decodes to the first latent vector Z, and then each element Z₁, Z₂, Z₃ . . . of the first latent vector Z is decoded by its respective individual first decoder 208p1, 208p2, 208p3 . . . For instance, a random or unobserved value of the latent vector H can be input to the second decoder 208pH in order to generate a new instance of the feature vector \hat{X} that was not observed in the training data. E.g. this could be used to generate a fictional face for use in a move or game, or to generate details of a functional patient for training or study purposes, etc.

FIG. 5E illustrates another example, where a third decoder network 208pY is trained during the second training stage to decode the second latent vector H into a classification Y. During the second training stage, each data point (each instance of the input feature vector Xo) is labelled with a corresponding observed value of the classification Y. Based on this, the learning function 209 trains the third decoder 208pY (i.e. tunes its weights) so as to minimize a measure of difference between the labelled classification and the predicted classification. After training, an unlabelled input feature vector Xₒ can then be input into the second encoder 208qH of the model 208′, in order to generate a corresponding prediction of the classification Y.

In another example, the model 208′ can be used to impute missing values in the input feature vector Xₒ. Following training, a subsequent observed instance of the feature vector Xₒ may be input to the second encoder 208qH, wherein this instance of the feature vector Xₒ which has some (but not all) of the features (i.e. elements) of the feature vector missing (i.e. unobserved). The missing elements may be set to zero, 50% or some other predetermined value representing “no observation”. The value(s) of the corresponding features (i.e. same elements) of the feature space can then be read out from the decoded version \hat{X} of the feature vector, and taken as imputed values of the missing observations. In embodiments, the model 208′ may also be trained using some data points that have one or more missing values.

An issue with this basic method of imputation is that the predetermined value representing “no observation” may still be interpreted by the encoder as if it was a sampled value. E.g. if 0 is used, then the encoder cannot tell the difference between “no observation” and an actual observation of zero (e.g. a black pixel, or a sensor reading of zero, etc.). Similar issues may apply if, say, a predetermined value of 50% probability is used.

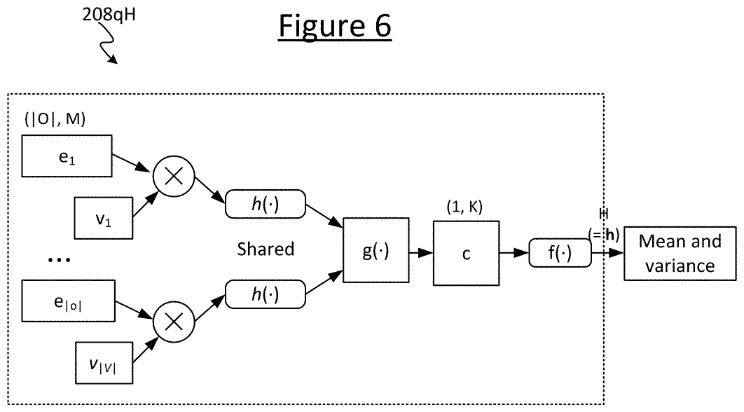

FIG. 6 shows an example structure of the second encoder 208qH which can be used to improve the imputation of missing features. In FIG. 6, each function h(⋅) represents an individual constituent neural network, each taking a respective input (N.B. h(⋅) as a function is not to be confused with h the latent vector discussed later—also called H above). Each input is a combination (e.g. multiplication) of a respective embedding e with a respective value v, where v is either X_{d} or Z_{d}. Preferably as many values of X and Z are input as available, during training and/or imputation. However, for any given data point during training and/or imputation the value of X_{d} and/or Z_{d} may be missing for some feature d (if X_{d} is missing then Z_{d} will also be missing). For any values not present, the corresponding inputs are simply omitted (there is no need to replace it with an input of a predetermined value like 0 or 50%). This is possible because of the use of the permutation invariant operator g(⋅), discussed shortly.

Each value v is combined with its respective embedding, e.g. by multiplication or concatenation, etc. In embodiments multiplication is used here, but it could be any operator that combines the information from the two. The embedding e is the coordinate of the respective input—it tells the encoder which element is being input at that input. E.g. this could be a coordinate of a pixel or an index of the feature d.

Each individual neural network h(⋅) outputs a vector. These vectors are combined by a permutation invariant operator g, such as a summation. A permutation invariant operator is an operator which outputs a value—in this case a vector—which depends on the values of the inputs to the operator but which is independent of the order of those inputs. Furthermore, this output vector is of a fixed size regardless of the number of inputs to the operator. This means that g(⋅) can supply a vector c of a given format regardless of which inputs are present and which are not, and the order in which they are supplied. This enables the encoder 208qH to deal handle missing inputs.

The encoder 208qH comprises a further, common neural network f(⋅) which is common to all of the inputs v. The output c of the permutation invariant operator g(⋅) is supplied to the input of this further neural network f(⋅). This neural network encodes the output c of g(⋅) into the second latent vector H (also labelled h, as a vector rather than a function, in the later working). In embodiments the further neural network f(⋅) is used, rather than just using c directly, because the size of observed features are not fixed. Therefore first a common function f is preferably applied to all observed features.

In an optional additional application of the disclosed model, a reward function R₁ may be used to determine which observation to make next following the first and second stages of training of the model 208′. The reward function is a function of the observations obtained so far, and represents the amount of new information that would be added by observing a given missing input. By determining which currently missing feature maximizes the reward function (or equivalently minimizes a cost function), this determines which of the unobserved inputs would be the most informative input to collect next. It represents the fact that some inputs have a greater dependency on one another than others, so the input that is least correlated with the other, already-observed inputs will provide the most new information. The reward function is evaluated for a plurality of different candidate unobserved features, and the feature which maximises the reward (or minimizes the cost) will be the feature that gives the most new information by being observed next. In some cases the model 208′ may then undergo another cycle of the first and second training stage, now incorporating the new observation. Alternatively the new observation could be used to improve the quality of a prediction, or simply be used by a human analyst such as a doctor in conjunction with the result (e.g. classification Y or an inputted missing feature X_{d}) of the already-trained model 208′.

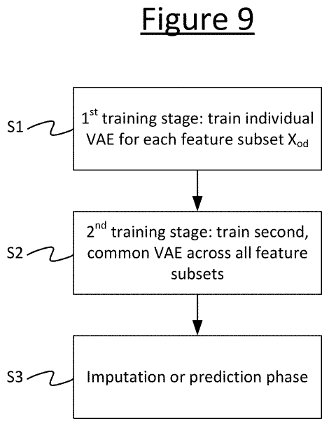

FIG. 9 is a flow chart giving an overview of a method in accordance with the presently disclosed approach. At step S1 the first training stage is performed, in which each of the individual VAEs is trained separately for a respective one of the feature subsets in order to learn to encode for that feature subset in a manner that is disentangled for the other feature subsets, i.e. to model the marginal properties of each feature subset. At step S2 the second training stage is performed, in which the second, common VAE is trained to learn or model the dependencies between feature subsets. At step S3 the trained model 208′ may be used for a practical purpose such as to make a prediction or perform an imputation. In an optional additional step, not shown—either between S2 and S3, or after S3—the reward function may be used to determine which missing feature to observed next. In some cases the method may then comprise observing this missing feature and cycling back to S1 to retrain the model 208′ including the new observation of the previously missing feature.

Note that while examples herein have been described as using labelled training data, the disclosed techniques are not limited to a supervised approach. More generally “training” herein could refer to any of supervised, reinforcement or unsupervised learning. The disclosed method is a specific way to obtain a model that can model datasets with mixed-type variables. Once the model is trained, it can be used in many ways such as reinforcement learning and prediction.

Some example implementation details of various concepts discussed above will now be discussed further by way of illustration.

In order to properly handle the mixed type data with heterogeneous marginals, the proposed method fits the data in a two-stage procedure. As shown in FIG. 5C, in the first Stage, for each variable we fit a low-dimensional VAE independently to each marginal data distribution. We call them marginal VAEs. Then, in the second stage, in order to capture the inter-variable dependencies, a new multi-dimensional VAE, called the dependency network, is built on top of the latent representations provided by encoders in the first Stage. We use D to denote the dimension of the observations and N is the number of data points x, thus x_{nd} is the dth dimension of the nth point. We present the details below.

Stage one: training individual marginal VAEs to each single variable. In the first stage, we focus on modelling the marginal distributions of each variable, by training D individual VAEs p_{θ}_{_{d}}(x_{nd})= [symbol] ₚ(z_{d})p_{θ}_{_{d}}(x_{nd}|z_{nd}), ∀d∈{1, 2, . . . , D} independently, each one is trained to fit a single dimension x_{d} from the dataset:

$$( {θ}_{d}^{*} , {ϕ}_{d}^{*} ) = arg \{ {max}_{{θ}_{d} , {ϕ}_{d}} {\sum }_{n} {𝔼}_{{q}_{{ϕ}_{d}} ( {z}_{nd} \vert {x}_{nd} )} \cdot log \frac{{p}_{{θ}_{d}} ( {x}_{nd} , {z}_{nd} )}{{q}_{{ϕ}_{d}} ( {z}_{nd} \vert {x}_{nd} )} \} ∀ d \\ ∈ \{ 1 , 2 , … , D \} , \qquad Eq . 1$$

where p(z_{d}) is the standard Gaussian prior, q_{ϕ}_{_{d}}(z_{nd}|x_{nd}) is the Gaussian encoder of the d-th marginal VAE. To specify the likelihood terms p_{θ}_{_{d}}(x_{nd}|z_{nd}), we use Gaussian likelihoods for continuous data, categorical likelihood with one-hot representation for categorical data. Note that the equation Eq. 1 Eq. 2 contains D independent objectives. Each VAE p_{d}(x_{nd}; θ_{d}) is trained independently and is only responsible for modelling the individual statistical properties of a single dimension x_{d} from the dataset. Thus, we assume that z_{d} is a scalar without loss of generality, although there is no limitation for its dimensionality. Each marginal VAE can be trained independently until convergence, hence avoiding the optimization issues of vanilla VAEs. We then fixed the parameters of marginal VAEs O_{d}*.

Stage two: training dependency network to assemble marginal VAEs. In the second stage, we model the intervariable statistical dependencies by training a new multi-dimensional VAE p_{ψ}(z)= [symbol] ₚ₍ₕ₎p_{ψ}(z|h), called the dependency network, is built on top of the latent representations z provided by encoders of marginal VAEs in first Stage. Specifically, we train p_{ψ}(z) by:

$$\begin{matrix}{x}_{data} \text{∼} {p}_{data} ( x ) & Eq . 2 \\ {z}_{d} \text{∼} {q}_{𝒫 d} ( {z}_{d} \vert {x}_{data , d} ) ∀ d ∈ \{ 1 , … , D \} & Eq . 3 \\ Δ ( ψ , λ ) ∝ {Δ}_{( ψ , λ )} {𝔼}_{qλ ( h \vert z , {x}_{data} )} log \frac{p ψ ( z , h )}{q λ ( h \vert z , {x}_{data} )} & Eq . 4\end{matrix}$$

where h is the latent space of the dependency network. The above procedure effectively disentangles the inter-variable, heterogeneous properties of mixed type data (modelled by marginal VAEs), from inter-variable dependencies (modelled by prior networks). We call our model VAE for heterogeneous mixed type data (VAEM).

After training the marginal VAEs and dependency network, our final generative model is given by:

$${p}_{θ} ( x ) = {𝔼}_{( z , h ) ∼ p ( h ) {Π}_{d} {p}_{ψ} ( {z}_{d} \vert h )} [ {\prod }_{d} {p}_{{θ}_{d}} ( {x}_{d} \vert {z}_{d} ) ] \qquad Eq . 5$$

To handle complicated statistical dependencies, we utilize the VampPrior, which uses a mixture of Gaussians (MoGs) as the prior distribution for the high-level latent variable i.e.,

$$p ( h ) = \frac{1}{K} {\sum }_{k} q λ ( h \vert {u}_{k} ) ,$$

where K<<N and uₖ are a subset of points.

In generic machine learning applications, normalization is considered to be an essential preprocessing step. For example, it is common to first normalize the data to have the zero mean and standard deviation. However, for mixed-type data, no standard normalization method can be applied. With our VAEM, each marginal VAE is trained independently to model the heterogeneous properties of each data dimension, thus transforming the mixed type data x_{d} to a continuous representation z_{d}. The collection of z_{d} forms the aggregated posterior which is close to a standard normal distribution thanks to the regularization effect from the prior p(z). In this way, we overcome the heterogeneous mixed-type problem and the dependency VAE can focus on learning the relationships among variables.

We further extend our method for decision making under uncertainty. In particular, we focus on the sequential active information acquisition application as an exemplar case. With this application context, we present the extension of using our model in the presence of missing data and Lindley information estimation.

Suppose that for a data instance x, we are interested in predicting a target x_{Φ}∈x_{U} of interest given currently observed x_{O}(x_{ϕ}∩x_{O}=ø), where x_{O} denotes the set of currently observed variables, and x_{U} the unobserved ones. One important problem is sequential active information acquisition (SAIA): how can we decide which variable xᵢ⊂x_{UΦ} is the best one to observe next, so that we can optimally increase our knowledge (e.g., predictive ability) regarding x_{Φ}?

To solve the problem:

1) we should have a good generative model that can handle missing data, and which can effectively generate conditional samples from log p(x_{U}|x_{O}),
2) the ability to estimate a reward function, in this case Lindley information, to enable decision making based on generative models. We now present our extensions of VAEM to fulfil these two requirements.

The amortized inference approach of VAEM cannot handle partially observed data, since the dimensionality of observed variables xo might vary across different data instances. We apply a PointNet encoding structure to build a partial inference network for the dependency VAE to infer h based on partial observations in an amortized manner. Specifically, at the first stage, we estimate each marginal VAE with only the observed samples for that dimension.

$$( {θ}_{d}^{⋆} , {ϕ}_{d}^{⋆} ) = arg \{ {max}_{θd , ϕd} ( {x}_{nd} ) {𝔼}_{{q}_{{ϕ}_{d}}} ( {z}_{nd} {x}_{nd} ) \cdot log \frac{{p}_{{θ}_{d}} ( {x}_{nd} , {z}_{nd} )}{{q}_{{ϕ}_{d}} ( {z}_{nd} \vert {x}_{nd} )} \} ∀ d ( 7 ) \\ ∈ \{ 1 , 2 , D \} , \qquad Eq . 6$$

where [symbol] ₓ_{ₙ}_{,O}(x_{nd})=1 iff x_{nd} ∈x_{n,O}, and x_{n,O} is the observed variables for the n-th data instance.

At the second stage, a VAE which can handle partial observation is needed. Similarly to the partial-VAE, the dependency VAE in the presence of missing data is defined by

$${p}_{{z}_{O}} ( {z}_{O} ; ψ ) = {E}_{p ( h )} {\prod }_{d ∈ O} {p}_{ψ} ( {z}_{d} \vert h ) p ( h ) \qquad Eq . 7$$

This is trained by maximising the partial ELBO:

$${z}_{d} \text{∼} {q}_{d} ( {z}_{d} \vert {x}_{data , d} , {ϕ}_{d} ) ∀ d ∈ O , {z}_{O} = \{ {z}_{d} \vert d ∈ O \} Eq . 8$$

$$Δ ( ψ , λ ) ∝ {∇}_{( ψ , λ )} {𝔼}_{q λ ( h \vert {z}_{O ,} {x}_{O} )} log \frac{{\prod }_{d ∈ O} {p}_{ψ} ( {z}_{d} \vert h ) p ( h )}{{q}_{λ} ( h \vert {z}_{O} , {x}_{O} )} Eq . 9$$

where h is the latent space of the dependency network, q_{λ}(h|z_{O}, x_{O}) is a set-function the so-called partial inference net, the structure of which is shown in FIG. 6. Essentially, for each data instance x_{O}, the input to the partial inference net is first modified as sₒ:={νe_{ν}|ν∈z_{O}∪x_{O}) using element-wise multiplication, and e_{ν} is a feature embedding. s_{O} is then fed into a feature map (a neural network) l(⋅): [symbol] ^{M}→ [symbol] ^{K}, where M and K is the [symbol] dimension of the feature embedding and the feature map, respectively. Finally, we apply a permutation invariant aggregation operation g(.), such as summation. In this way, q_{λ}(h|z_{O}, x_{O}) is invariant to the permutations of elements of x_{O}, and x_{O} can have arbitrary length.

Once the marginal VAEs and the partial dependency network is trained, we can generate conditional samples from log p_{θ}(x_{U} x_{O}) by the following inference procedure: first, the latent representation z_{d} for the observed variables are inferred. With this representation, we utilize the partial inference network to infer the h, which is the latent code for the second stage VAE. With the h, we can generate zₛ which are the latent code for the unobserved dimensions and then generate the xₛ.

z_{d}˜q_{d}(z_{d}|x_{d},ϕ_{d})∀d∈O, z_{O}=z_{d}|d∈O Eq. 10

h˜q_{λ}(h|z_{O},z_{O}) Eq. 11

zₛ˜p_{ψ}(zₛ|h)∀s∈U, z_{U}={zₛ|s∈U} Eq. 12

xₛ˜p_{θ}(xₛ|z_{U},z_{O})∀s∈U, x_{U}=xₛ|s∈U Eq. 13

SAIA can be framed as a Bayesian experimental design problem. xᵢ⊂x_{UΦ} is selected by the following information reward function

R_{I}(xᵢ,xₒ)= [symbol] ₓ_{ᵢ}_{˜p}(xᵢ|x_{O}) [symbol] [p(x_{Ψ}|x_{O})] Eq. 14

We use a pre-trained partial VAEM model to estimate the required distributions p(xᵢ|x_{O}), p(xψ|xᵢ,x_{O}), and p(xΦ|x_{O}). Due to the intractability of [symbol] [p(x_{Φ}(xᵢ,x_{O})∥p(x_{Φ}|x_{O})], we must resort to approximations. An efficient latent space estimation method of R_{I}(xᵢ, x_{O}) can be approximated by:

[symbol]

(xᵢ,x_{O})= [symbol] _{pθ(x}_{ⱼ}_{,z}_{ᵢ}_{,z}_{_{O}}_{|x}_{_{O}}₎{ [symbol] [q_{λ}(h|z_{O})]− [symbol] _{(pθx}_{_{ψ}}_{,z}_{_{ϕ}}_{,x}_{_{O}}₎ [symbol] [q_{λ}(h|z_{Φ},zᵢ,z_{O})q_{λ}(h|z_{Φ},z_{O})}] Eq. 15

Note that for compactness, we omitted the notation for input xₒ and xᵢ to the partial inference nets. The approximation is very efficient to compute, since all KL terms can be calculated analytically, assuming that the partial inference net q_{λ}(h|z_{O}) is Gaussian (or other common distributions such as normalising flows).

In active information acquisition, the target of interest xΦ∩x_{O}=Ø is often the target that we try to predict. In order to enhance the predictive performance of VAEM, we propose to use the following factorization:

p_{θ}(x_{O},x_{Φ})= [symbol] _{pθ(x}_{_{U}}_{Φ,h|x}_{_{O}}₎p_{λ}(x_{Φ}|x_{O},x_{UΦ},h)p_{θ}(x_{O}) Eq. 16

where p_{λ}(xΦ|x_{O}, x_{UΦ}) is the discriminator that gives probabilistic prediction of x_{Φ} based on both observed variables x_{O}, imputed variables x_{U} and the global latent representation h (the last one is optional). The discriminator in Equation Eq. 16 offers additional predictive power for the target x_{Φ} of interest.

To evaluate the performance and validity of our proposed VAEM model, we first assess it on the task of mixed type heterogeneous data generation. Then, we compare the performance of conditional mixed type data generation (imputation). Finally, to evaluate the conditional generation quality of our models more comprehensively, we apply VAEM to the task of sequential active information acquisition. In these tasks, the underlying generative model is asked to generate samples of unobserved variables for each instance, and then decide which variables to acquire next. The same set of datasets are used for all experiments, which include two UCI benchmark datasets (Boston housing and Energy), two real-world datasets Avocado and Bank, and medical dataset (MIMIC-III). We compare our proposed VAEM with a number of baseline methods.

For our proposed method VAEM, unless specified, we use the partial version proposed above, and the discriminator structure specified by equation Eq. 16

TABLE 1
Data generation quality by test NLL per variable, with standard errors as error bars.

| Method | Ours | VAE | VAE-balanced | VAE-extended | VAE-HI |
|---|---|---|---|---|---|
| Bank | −1.15 ± 0.09 | 2.09 ± 0.04 | 0.72 ± 0.01 | 2.06 ± 0.00 | −0.72 ± 0.00 |
| Boston | −2.16 ± 0.01 | −1.69 ± 0.01 | 0.38 ± 0.01 | −1.61 ± 0.02 | 2.11 ± 0.01 |
| Avocado | −0.16 ± 0.00 | 0.04 ± 0.00 | 1.32 ± 0.01 | 0.04 ± 0.00 | 0.04 ± 0.00 |
| Energy | −1.28 ± 0.09 | −1.47 ± 0.07 | 0.69 ± 0.02 | −1.46 ± 0.08 | 0.16 ± 0.00 |
| MIMIC | −1.01 ± 0.00 | 0.08 ± 0.00 | 0.69 ± 0.00 | 0.08 ± 0.00 | 0.08 ± 0.00 |
| Avg. Rank | 1.40 ± 0.36 | 2.60 ± 0.61 | 4.40 ± 0.36 | 3.00 ± 0.40 | 3.00 ± 0.57 |

Throughout the experiments, we consider a number of baselines. Unless specified, all VAE baselines also use similar partial inference method and discriminator structure. Moreover, all baselines are equipped with MoG priors. Our main baselines include:
Heterogeneous-Incomplete VAE (HI-VAE). We adopt the multi-head structure of HI-VAE and match the hidden unit dimensionalities to be the same as our VAEM. HI-VAE is an important baseline, since it is motivated in a similar way to our VAEM, but trained in an end-to-end manner rather than two-stage. We denote this by VAE-HI.
VAE: A vanilla VAE equipped with VampPrior. The number of latent dimensions is the same as that in the second stage of VAEM. We denote this by VAE.
VAE with extended latent dimensions: Note that the total latent dimension of VAEM is D+L, where D and L is the dimensionalities of the data instance and h respectively. To be fair, in this baseline we extend the latent dimension of vanilla VAE baseline to D+L. We denote this baseline by VAE-extended.
VAE with automatically balanced likelihoods. This baseline tries to automatically balance the scale of the log-likelihood values of different variable types in the ELBO, by adaptively multiplying a constant before likelihood terms We denote this baseline by VAE-balanced.

We use the same set of mixed type datasets for all tasks. They include:
Two standard UCI benchmark datasets: Boston housing (13 continuous, 1 categorical) and energy efficiency (6 continuous, 3 categorical);
Two relatively large real-life dataset: Bank marketing dataset (45211 instances, 11 continuous, 8 categorical, 2 discrete); and Avocado sales prediction dataset (18249 instances, 9 continuous, 5 categorical)
One real-world medical dataset: Medical Information Mart for Intensive Care (MIMIC III) database, the largest public medical dataset containing records of 21139 patients (We mainly focus on the mortality prediction task based on 17 medical instruments (13 continuous, 4 categorical). Since the dataset is imbalanced (over 80% of the data has mortality=0), we balance the dataset by down-sampling to better demonstrate the model behaviors. All time-series variables are averaged to give static features.

### Mixed Type Data Generation Task

In this task, we evaluate the quality of our generative model in terms of mixed type data generation. During training, the range of all variables all scaled to the range between 0 and 1. For all datasets, we first train the models and quantitatively compare their performance on test set using a 90%-10% train-test split. All experiments are repeated 5 times over different random seeds.

Visualization by pair plots: For deep generative models, the data generation quality reflects how well the model models the data. Thus, we first visualize the data generation quality of each model on a representative dataset, Bank marketing. Bank dataset containing three different data types, each with drastically different marginals which present challenges for learning. We fit our models to Bank dataset, and then generate the pair plots for three of the variables, x₀, x₁ and x₂ (first two are categorical, the third one is continuous) selected from the data (FIG. 7). In each subfigure of FIG. 7, diagonal plots shows the marginal histograms of each variable. The upper-triangular part shows sample scatter plots of each variable pair. The lower-triangular part shows heat maps identifying regions of high-probability density for each variable pair. FIG. 7 (a) shows the ground truth for each variable. FIG. 7 (b) shows the values calculated using VAEM. FIG. 7 (c) shows the values calculated by vanilla VAE. FIG. 7 (d) shows the values calculated used VAE-extended. FIG. 7 (e) shows the values calculated using VAE-balanced. FIG. 7 (f) shows the values calculated using VAE-HI.

The vanilla VAE is able to generate the second categorical variable. However, note that the third variable of the dataset (FIG. 7 (a)), which corresponds to the “duration” feature of the dataset, is a very important variable that has a heavy tail. The vanilla VAE (FIG. 7 (c)) fails to mimic this heavy tail behaviour of the variable. On the other hand, although the VAE-balanced model and VAE-HI (FIG. 7(e) (f)) can capture part of this heavy-tail behaviour, it fails to model the second categorical variable well. Our VAEM model (FIG. 7 (b)) is able to generate accurate marginals and joint distributions for both categorical and heavy-tailed continuous distribution.

Quantitative evaluation on all datasets: To evaluate the data generation quality quantitatively, we compute the marginal negative log-likelihood (NLL) of the models on test set. Note that all NLL numbers are divided by the number of variables of the dataset. As shown in Table 1, VAEM can consistently generate realistic samples, and on average significantly outperforms other baselines.

### Mixed Type Conditional Data Generation Task

An important aspect of generative models is the ability to perform conditional data generation. That is, given a data instance, to infer the posterior distribution regarding unobserved variables x_{U} given x_{O}. For all baselines evaluated in this task, we train the partial version of them (i.e., generative+partial inference net). To train the partial models, we randomly sample 90% of the dataset to be training set, and remove a random portion (uniformly sampled between 0% and 99%) of observations each epoch during training. Then, we remove 50% of the test set and use generative models to make inference regarding unobserved data. Since all inference are probabilistic, we report the negative test NLLs on unobserved data, as opposed to imputation RMSE typically used in the literature.

Results are summarized in Table 2, where all NLL values have been divided by the number of observed variables. We repeat our experiments for 5 runs and report standard errors. Note that how the automatic balancing strategy—VAE-balanced almost always makes the performance worse. On the contrary, Table 2 shows that our proposed method is very robust, yielding significantly better performance than all baselines on 4 out of 5 datasets, and competitive performance on energy dataset.

TABLE 2

| Method | Ours | VAE | VAEbalanced | VAEextended | VAE-HI |
|---|---|---|---|---|---|
| Bank | −1.21 ± 0.12 | 2.09 ± 0.00 | 0.68 ± 0.00 | 2.09 ± 0.00 | −0.83 ± 0.01 |
| Boston | −2.18 ± 0.03 | −1.66 ± 0.02 | 0.37 ± 0.00 | −1.67 ± 0.01 | 1.58 ± 0.01 |
| Avocado | −0.15 ± 0.00 | 0.04 ± 0.00 | 1.33 ± 0.00 | 0.04 ± 0.00 | 0.04 ± 0.00 |
| Energy | −1.30 ± 0.05 | −1.50 ± 0.06 | 0.67 ± 0.01 | −1.50 ± 0.06 | 0.13 ± 0.00 |
| MIMIC | −1.10 ± 0.00 | 0.08 ± 0.00 | 0.57 ± 0.00 | 0.08 ± 0.00 | 0.08 ± 0.00 |
| Avg. Rank | 1.40 ± 0.36 | 2.60 ± 0.61 | 4.40 ± 0.38 | 2.30 ± 0.44 | 3.00 ± 0.57 |

In our final experiments, we apply VAEM to the task of sequential active information acquisition (SAIA) based on the formulation described above. We use this task as an example to showcase how VAEM can be used in decision making under uncertainty. In SAIA, at each step the underlying generative model is asked to generate posterior samples of unobserved variables x_{U} for each data instance x, and then decide which variables to acquire next. SAIA is a perfect task for evaluating generative models on mixed type data, since it integrates data generation, conditional generation, target prediction and decision making into a single task. Deep generative models with efficient inference that can handle partial observations are essential components for SAIA task.

We first pre-train our models and baselines according to the settings outlined above. Then, in SAIA, we actively select variable for each test instance starting with empty observation x_{O}=ø. The reward function of VAEM is estimated as described above. We add an additional baseline, denoted by VAE-no-disc, which is a VAE without discriminator structure. This is the baseline to show the importance of the extension described above in prediction tasks. Other settings are the same as VAE baseline. All experiments are repeated for ten times.

FIG. 8 shows the test RMSEs on x_{Φ} for each variable selection step on all five datasets, where xΦ is the target variable of each dataset. The y-axis shows the error of the prediction and the x-axis shows the number of features acquired for making the prediction. We call the curve in FIG. 8 as the information curves, where the area under the information curves (AUIC) can be used to evaluate the performance of SAIA of a given model. Smaller area is desired. From FIG. 8, we see that VAEM performs consistently better than other baselines. Note that on Bank marketing and Avocado sales datasets where a lot of heterogeneous variables are involved, other baselines almost fails to reduce test RMSE quickly, where VAEM outperforms them by a large margin. These experiments show that VAEM is able to acquire information efficiently on mixed type datasets.

It will be appreciated that the above embodiments have been described by way of example only.

More generally, according to one aspect disclosed herein, there is provided a method comprising: in a first stage, training each of a plurality of individual first variational auto encoders, VAEs, each comprising an individual respective first encoder arranged to encode a respective subset of one or more features of a feature space into an individual respective first latent representation having one or more dimensions, and an individual respective first decoder arranged to decode from the respective latent representation back to a decoded version of the respective subset of the feature space, wherein different subsets comprise features of different types of data; and in a second stage following the first stage, training a second VAE comprising a second encoder arranged to encode a plurality of inputs into a second latent representation having a plurality of dimensions, and a second decoder arranged to decode the second latent representation into decoded versions of the first latent representations, wherein each respective one of the plurality of inputs comprises a combination of a different respective one of feature subsets with the respective first latent representation.

As the auto encoders are variational auto encoders, each dimension of their latent representation is modelled as a probabilistic distribution. In embodiments, the decoded versions of the features as output by the decoders may also be modelled as distributions, or may be simple scalars. The weights of the nodes in the neural networks may also be modelled as distributions or scalars.

Each of the encoders and decoders may comprise one or more neural networks. The training of each VAE may comprise comparing the features as output by the decoder with the features as input to the encoder, and tuning parameters of the nodes of the neural networks in the VAE to reduce the difference therebetween.

In embodiments, each of said subsets is a single feature.

Alternatively, in embodiments, each of said subsets may be more the one feature. In this case the respective features within each subset may be of the same type but a different respective data type relative to the other subset.

In embodiments, each of the first latent representations is a single respective one-dimensional latent variable.

Note again however that as the auto encoders are variational auto encoders, each latent variable is nonetheless still modelled as a distribution.

In embodiments, the different data types may comprise two or more of: categorical, ordinal, and continuous.

In embodiments, the different data types may comprise: binary categorical, and categorical with more than two categories.

In embodiments, the features may comprise one or more sensor readings from one or more sensors sensing a material or machine.

In embodiments, the features may comprise one or more sensor readings and/or questionnaire responses from a user relating to the users health.

In embodiments, a third decoder may be trained to generate a categorization from the second latent representation.

In embodiments, the second encoder may comprise a respective individual second encoder arranged to encode each of a plurality of the feature subsets and/or first latent representations, a permutation invariant operator arranged to combine encoded outputs of the individual second encoders into a fixed size output, and a further encoder arranged to encode the fixed size output into the second latent representation.

In embodiments, said combination may be a concatenation.

Aspects disclosed herein also provide a method of using the second VAE, after having been trained as hereinabove mentioned in any of the aspects or embodiments, to perform a prediction or imputation.

In embodiments, the method may use the second VAE to predict or impute a condition of the material or machine.

In embodiments, the method may use the second VAE to predict or impute a health condition of the user.

In embodiments, the method may use the third decoder together with the second encoder, after having been trained, to predict the categorization of a subsequently observed feature vector of said feature space.

In embodiments the method may use the second VAE, after having been trained, to impute a value of one or more missing features in a subsequently observed feature vector of said feature space, by:
supplying observed values of the feature vector as values of the features of the respective inputs to the second encoder,
setting each unobserved feature in said inputs to a predetermined value representing no observation, and
reading values of features of said feature space, as output by the first decoders, corresponding to the unobserved features.

In embodiments the method may use the second encoder after having been trained, to impute one or more unobserved features by:
supplying observed values of the feature vector as values of the features of the respective inputs to the second encoder, omitting the inputs corresponding to the one or more unobserved features,
using the permutation invariant operator to convert the remaining observed features into the fixed size output of the same size as during training,
supplying the resulting first latent representations into the respective first decoders, having been trained during the first training stage, and
reading values of features of said feature space, as output by the first decoders, corresponding to the unobserved features.

Another aspect provides a computer program embodied on computer-readable storage and configured so as when run on one or more processing units to perform the method of any of the aspects or embodiments hereinabove defined.

Another aspect provides a computer system comprising: memory comprising one or more memory units, and processing apparatus comprising one or more processing units; wherein the memory stores code arranged to run on the processing apparatus, the code being configured so as when run on the processing apparatus to carry out the method of any of the aspects or embodiments hereinabove defined.

In embodiments, the computer system may be implemented as a server comprising one or more server units at one or more geographic sites, the server arranged to perform one or both of:
gathering observations of said features from a plurality of devices over a network, and using the observations to perform said training; and/or
providing prediction or imputation services to users, over a network, based on the second VAE once trained.

In embodiments the network for the purpose of one or both of these services may be a wide area internetwork such as the Internet. In the case of gathering observations, said gathering may comprise gathering some or all of the observations from a plurality of different users through different respective user devices. As another example said gathering may comprise gathering some or all of the observations from a plurality of different sensor devices, e.g. IoT devices or industrial measurement devices.

Another aspect provides use of a variational encoder which has been trained by, in a first stage, training each of a plurality of individual first variational auto encoders, VAEs, each comprising an individual respective first encoder arranged to encode a respective subset of one or more features of a feature space into an individual respective first latent representation having one or more dimensions, and an individual respective first decoder arranged to decode from the respective latent representation back to a decoded version of the respective subset of the feature space, wherein different subsets comprise features of different types of data; and in a second stage following the first stage, training a second VAE comprising a second encoder arranged to encode a plurality of inputs into a second latent representation having a plurality of dimensions, and a second decoder arranged to decode the second latent representation into decoded versions of the first latent representations, wherein each respective one of the plurality of inputs comprises a combination of a different respective one of feature subsets with the respective first latent representation, the use being of the second variational encoder.

In example applications, the trained model may be employed to predict the state of a condition of a user, such as a disease or other health condition. For example, once trained, the model may receive the answers to questions presented to a user about their health status to provide data to the model. A user interface may be provided to enable questions to be output to a user and to receive responses from a user for example through a voice or other interface means. In some example, the user interface may comprise a chatbot. In other examples, the user interface may comprise a graphical user interface (GUI) such as a point and click user interface or a touch screen user interface. The trained algorithm may be configured to generate an overall score from the user responses, which provide his or her health data, to predict a condition of the user from that data. In some embodiments, the model can be used to predict the onset of a certain condition of the user, for example, a health condition such as asthma, depression or heart disease.

A user's condition may be monitored by asking questions which are repeated instances of the same question (asking the same thing, i.e. the same question content), and/or different questions (asking different things, i.e. different question content). The questions may relate to a condition of the user in order to monitor that condition. For example, the condition may be a health condition such as asthma, depression, fitness etc. The monitoring could be for the purpose of making a prediction on a future state of the user's condition, e.g. to predict the onset of a problem with the user's health, or for the purpose of information for the user, a health practitioner or a clinical trial etc.

User data may also be provided from sensor devices, e.g. a wearable or portable sensor device worn or carried about the user's person. For example, such a device could take the form of an inhaler or spirometer with embedded communication interface for connecting to a controller and supplying data to the controller. Data from the sensor may be input to the model and form part of the patient data for using the model to make predictions.

Contextual metadata may also be provided for training and using the algorithm. Such metadata could comprise a user's location. A user's location could be monitored by a portable or wearable device disposed about the user's person (plus any one or more of a variety of known localisation techniques such as triangulation, trilateration, multiliteration or finger printing relative to a network to known nodes such WLAN access points, cellular base stations, satellites or anchor nodes of a dedicated positioning network such an indoor location network).

Other contextual information such as sleep quality may be inferred from personal device data, for example by using a wearable sleep monitor. In further alternative or additional examples, sensor data from e.g. a camera, localisation system, motion sensor and/or heart rate monitor can be used as metadata.

The model may be trained to recognise a particular disease or health outcome. For example, a particular health condition such as a certain type of cancer or diabetes may be used to train the model using existing feature sets from patients. Once a model has been trained, it can be utilised to provide a diagnosis of that particular disease when patient data is provided from a new patient. The model may make other health related predictions, such as predictions of mortality once it has been trained on a suitable set of patient training data with known mortality outcomes.

Another example of use of the model can be to determine geological conditions, for example for drilling to establish the likelihood of encountering oil or gas, for example. Different sensors may be utilised on a tool at a particular geographic location. The sensors could comprise for example radar, lidar and location sensors. Other sensors such as the thermometers or vibration sensors may also be utilised. Data from the sensors may be in different data categories and therefore constitute mixed data. Once the model has been effectively trained on this mixed data, it may be applied in an unknown context by taking sensor readings from equivalent sensors in that unknown context and used to generate a prediction of geological conditions.

A possible further application is to determine the status of a self-driving car. In that case, data may be generated from sensors such as radar sensors, lidar sensors and location sensors on a car and used as a feature set to train the model for certain condition that the car may be in. Once a model has been trained, a corresponding mixed data set may be provided to the model to predict certain car conditions.

A further possible application of the trained model is in machine diagnosis and management in an industrial context. For example, readings from different machine sensors including without limitation, temperature sensors, vibration sensors, accelerometers, fluid pressure sensors may be used to train the model for certain breakdown conditions of a machine. Once a model has been trained, it can be utilised to predict what may have caused a machine breakdown once data from that machine has been provided from corresponding sensors.

A further application is in the context of predicting heat load and cooling load for different buildings. Attributes of a building may be provided to the model for training purposes, these attributes including for example surface area, wall area, roof area, height, orientation etc. Such attributes may be of a mixed data type. As an example, orientation may be a categorical data type and area may be a continuous data type. Once trained, the model can be used to predict the heating load or cooling load of a particular building once corresponding data has been supplied to it for a new building.

Other variants or use cases of the disclosed techniques may become apparent to the person skilled in the art once given the disclosure herein. The scope of the disclosure is not limited by the described embodiments but only by the accompanying claims.

1. A method comprising:
in a first stage, training each of a plurality of individual first variational auto encoders, VAEs, each comprising an individual respective first encoder arranged to encode a respective subset of one or more features of a feature space into an individual respective first latent representation having one or more dimensions, and an individual respective first decoder arranged to decode from the respective latent representation back to a decoded version of the respective subset of the feature space, wherein different subsets comprise features of different types of data; and
in a second stage following the first stage, training a second VAE comprising a second encoder arranged to encode a plurality of inputs into a second latent representation having a plurality of dimensions, and a second decoder arranged to decode the second latent representation into decoded versions of the first latent representations, wherein each respective one of the plurality of inputs comprises a combination of a different respective one of feature subsets with the respective first latent representation.

2. The method of claim 1, wherein each of said subsets is a single feature.

3. The method of claim 1, wherein each of said subsets is more the one feature, and wherein the respective features within each subset are of the same type but a different respective data type relative to the other subset.

4. The method of claim 1, wherein each of the first latent representations is a single respective one-dimensional latent variable.

5. The method of claim 1, wherein the different data types comprise two or more of:
categorical, ordinal, and continuous.

6. The method of claim 1, wherein the different data types comprise: binary categorical, and categorical with more than two categories.

7. The method of claim 1, wherein the features comprise one or more sensor readings from one or more sensors sensing a material or machine.

8. The method of claim 1, wherein the features comprise one or more sensor readings and/or questionnaire responses from a user relating to the user's health.

9. The method of claim 1, comprising training a third decoder to generate a categorization from the second latent representation.

10. The method of claim 1, wherein the second encoder comprises a respective individual second encoder arranged to encode each of a plurality of the feature subsets and/or first latent representations, a permutation invariant operator arranged to combine encoded outputs of the individual second encoders into a fixed size output, and a further encoder arranged to encode the fixed size output into the second latent representation.

11. The method of claim 1, wherein said combination is a concatenation.

12. A method of using the second VAE, after having been trained according to claim 1, to perform a prediction or imputation.

13. A method of using the second VAE, after having been trained according to claim 7, to predict or impute a condition of the material or machine.

14. A method of using the second VAE, after having been trained according to claim 8, to predict or impute a health condition of the user.

15. A method of using the third decoder together with the second encoder, after having been trained according to claim 9, to predict the categorization of a subsequently observed feature vector of said feature space.

16. A method comprising using the second VAE, after having been trained according to claim 1, to impute a value of one or more missing features in a subsequently observed feature vector of said feature space, by:
supplying observed values of the feature vector as values of the features of the respective inputs to the second encoder,
setting each unobserved feature in said inputs to a predetermined value representing no observation, and
reading values of features of said feature space, as output by the first decoders, corresponding to the unobserved features.

17. The method comprising using the second encoder, after having been trained according to claim 10, to impute one or more unobserved features by:
supplying observed values of the feature vector as values of the features of the respective inputs to the second encoder, omitting the inputs corresponding to the one or more unobserved features,
using the permutation invariant operator to convert the remaining observed features into the fixed size output of the same size as during training,
supplying the resulting first latent representations into the respective first decoders, having been trained during the first training stage, and
reading values of features of said feature space, as output by the first decoders, corresponding to the unobserved features.

18. A computer program embodied on computer-readable storage and configured so as when run on one or more processing units to perform the method of claim 1.

19. A computer system comprising:
memory comprising one or more memory units, and
processing apparatus comprising one or more processing units;
wherein the memory stores code arranged to run on the processing apparatus, the code being configured so as when run on the processing apparatus to carry out the method of claim 1.

20. The computer system of claim 19 implemented as a server comprising one or more server units at one or more geographic sites, the server arranged to perform one or both of:
gathering observations of said features from a plurality of devices over a network, and using the observations to perform said training; and/or
providing prediction or imputation services to users, over a network, based on the second VAE once trained.
